(19) United States
(12) Patent Application Publication
(10) Pub. No.: US 2004/0014031 A1
(43) Pub. Date: Jan. 22, 2004

(54) Inducible highly productive rAAV packaging cell-lines

(76) Inventors: Anna Salvetti, Nantes (FR); Gilliane Chadeuf, Nantes (FR); Jacques Tessier, Nantes (FR); Philippe Moullier, Basse-Goulaine (FR); Michael R. Linden, New York, NY (US); Peter Ward, New York, NY (US); Alberto Luis Epstein, Lyon (FR)

Correspondence Address: YOUNG & THOMPSON, 745 SOUTH 23RD STREET 2ND FLOOR, ARLINGTON, VA, 22202

(21) Appl. No.: 10/212,772

(22) Filed: Aug. 7, 2002

Related U.S. Application Data
(63) Continuation of application No. PCT/EP01/15418, filed on Dec. 6, 2001.
(60) Provisional application No. 60/251,576, filed on Dec. 7, 2000.

(51) Int. Cl. C12Q 1/70; C12P 19/34; C12N 15/85

(52) U.S. Cl. 435/5; 435/455; 435/91.1

(57) ABSTRACT

The present invention relates to an isolated nucleic acid sequence comprising a first DNA sequence comprising a cis-acting replication element (CARE) from an Adeno-Associated Virus (AAV), and a second DNA sequence operably linked to said CARE, wherein amplification of said isolated nucleic acid sequence occurs when said isolated nucleic acid sequence is integrated in the genome of a cell and said cell is contacted with a CARE-dependent replication unducer (CARE-DRI). It also relates to amplification methods using a CARE-dependent replication inducer (CARE-DRI) and packaging cell-lines wherein replication of the integrated rep and cap genes is inducible by a CARE-DRI.

### FIELD OF THE INVENTION

The present invention relates to improved packaging cell-lines and methods for the production of recombinant Adeno-Associated Viruses (rAAV). In particular, the invention discloses nucleic acid sequences derived from the genome of AAV-2, and which behave like a replication origin in the presence of AAV Rep proteins and a helper virus. These sequences can be used in a number of applications necessitating the over-expression of a gene in a cell.

### BACKGROUND AND PRIOR ART

Wild type Adeno-Associated Virus (wtAAV) is a naturally defective parvovirus which requires co-infection with a helper virus, such as Adenovirus or Herpesvirus, in order to establish a productive infection. The virus is not associated with any human disease and has been shown to have a broad host range of infection in vitro.

The ability of recombinant AAV vectors (rAAV) to transduce tissues in vivo leading to stable gene expression, together with their innocuousness, has contributed to the widespread use and development of these vectors. For example, recent studies have reported the partial and long-term correction of factor IX deficiency in affected dogs, prompting the clinical evaluation of rAAV in hemophilia patients.

As for other viral vectors, an important concern in using rAAV vectors is the efficient production of clinical grade rAAV preparation. Ideally, rAAV preparations for clinical use should be free of replicative viral particles, free of helper virus particles (even defective ones), and highly concentrated. A better knowledge in AAV molecular biology could possibly lead to improvements in the methods for the generation of clinical grade rAAV.

The genome of adeno-associated virus type 2 (AAV-2) is a linear single-stranded DNA molecule of 4,679 nucleotides containing two ORFs, rep and cap, flanked by 145 base inverted terminal repeats (ITRs). The rep genes are regulated by two promoters, p5 and p19, and encode four non-structural Rep proteins. Rep78 and Rep68 are involved in the replication of viral DNA and in the regulation of AAV gene expression. They have DNA-binding, helicase, and site-specific and strand-specific endonuclease activities. A Rep-binding element (RBS) Is present in the ITRs, in the p5 and p19 promoters, and also in the AAVS1 locus of human chromosome 19 (19q13.4), in which AAV-2 can specifically integrate. Rep78 and Rep68 can also bind to a degenerate RBS containing a minimum 8 base pairs (bp) core sequence that is present in as many as 200,000 copies in the human genome. The two smaller Rep proteins, Rep52 and Rep40, do not exhibit any DNA-binding activity but strongly stimulate single-stranded AAV DNA accumulation. Recently, Rep52, which possesses an ATPase-dependent helicase activity, has been suggested to be involved in the viral DNA packaging pathway. The cap gene encodes three capsid proteins VP1, VP2, and VP3, which are translated from a single transcript initiated at the p40 promoter.

AAV DNA replication is initiated by the folding of the ITR into a hairpin structure which provides a free 3′-OH end for the conversion of the single-stranded DNA genome into a duplex DNA molecule in which one of the ends of the molecule is covalently joined. A strand-specific cleavage converts this covalently joined end into an open duplex end. This step is mediated by binding of Rep78 and Rep68 proteins to the RBS and cleavage at the terminal resolution site (trs), which is present in the ITR, downstream the RBS. This step is followed by unwinding of the DNA hairpin and replication outward to generate a blunt-ended, double-stranded molecule. Further rounds of replication proceed following a strand displacement mechanism once the ITR has resumed a hairpin structure, thus providing a new 3′-OH end for elongation.

Besides their role in DNA replication, the ITRs are commonly defined as necessary and sufficient for DNA encapsidation. However, specific DNA packaging signals within the ITRs have not been identified and the precise mechanism of DNA encapsidation is as yet unknown. Recently, the specificity of the DNA encapsidation has been proposed to occur through protein-protein interaction between the Rep proteins bound to the AAV-2 genome via the RBS and those already associated with the capsid [1].

Because of all these properties, the ITRs are currently the unique viral sequences retained in recombinant AAV vectors (rAAV), in which the rep and cap genes are replaced by the transgene linked to its regulatory elements.

To undergo a productive infection AAV requires the presence of a helper virus, adenovirus or herpesvirus. The helper virus, for instance adenovirus, plays a role in nearly every step of the AAV life cycle by promoting AAV gene expression and DNA replication. The critical adenovirus factors involved in the helper effect are the products of the E1a, E1b, E4(orf6), E2a genes and the VA1 RNA. Among these early adenovirus proteins, the one encoded by the E2a gene, the DNA Binding Protein (DBP), was shown to be directly implicated in AAV DNA replication by stimulating the processivity of DNA polymerization, possibly by stabilizing single-strand templates for replication [2].

Like “wild-type” AAV, replication and packaging of rAAV vectors require helper activities. rAAV production hence requires transcomplementation of both the AAV functions, and the helper activities. For rAAV assembly, the Rep and Cap functions are supplied in trans using a construct harboring an ITR-deleted AAV genome. According to the standard procedure, rAAV production relies upon co-transfection of the vector and the rep-cap plasmid into adenovirus-infected cells.

As an alternative to rep-cap plasmid transfection, recent studies described the development of cell lines derived from Hela cells and harboring a vector and a rep-cap genome devoid of the AAV ITRs. In this configuration, the rep and cap genes are under the control of the native AAV promoters which are silent or only poorly active in the absence of adenoviral proteins. Upon adenoviral infection, these cell lines produce rAAV to a level similar to that obtained after transient transfection of a rep-cap plasmid into 293 cells [3]. An improvement in rAAV yields from stable HeLa packaging cell lines was reported in three recent studies. The first study used a HeLa cell clone in which the integrated rep-cap and vector sequences were conditionally amplified using an SV40-based system [4]. In two other studies, high yields of rAAV assembly were reported using a rep-cap HeLa cell clone infected with a hybrid adenovirus in which the AAV vector was cloned in the E1 region [5, 6]. More recently, the inventors have described a dramatic amplification of the rep-cap genome in HeRC32 cells, which are derived from HeLa cells and harbor an integrated rep-cap copy [7]. These cells have been deposited at the Collection Nationale de Cultures de Micro-organismes (CNCM) on 30^{th} May 2001, under the reference n° I-2675. Similar results have then been observed in other cell-lines [8]. Up to now, the factors involved in the rep-cap amplification in stable cell-lines have not been described, except for the involvement of adenovirus infection.

As an alternative to adenoviral infection in the standard rAAV production procedure, the adenoviral helper functions can be provided by transfecting a plasmid encoding the essential adenoviral functions. Under these conditions, the recombinant vector genome is rescued from the plasmid, replicated, and finally packaged into AAV capsids. However, despite numerous improvements introduced in this procedure, rAAV titers remain approximately 10- to 100-fold lower than those obtained for wild type AAV [9]. Moreover, the precise characterization of rAAV preparations using sensitive assays such as the Replication Center Assay (RCA), indicated that the vector stocks were contaminated to various extents with particles containing wild type AAV-like sequences. In a previous publication, the inventors called these contaminating particles, “rep-positive”, because they were able to transfer a Rep function, as detected by RCA [10]. Previous studies have shown that most of these particles were replication competent, as shown by serial amplification on adenovirus-infected cells, and that they arose from non-homologous recombination events between the rep-cap sequences and the ITRs present in the rAAV vector plasmid. Although deletion of critical ITR sequences involved in the non-homologous recombination events prevented the formation of such replication-competent AAV-like particles, rAAV preparations remained contaminated by AAV particles containing a rep-cap genome.

Both these observations, i.e. the low rAAV packaging efficiency and the generation of rep-positive AAV particles, suggested that some additional 15 elements involved in viral DNA replication and/or encapsidation were missing in rAAV vectors.

### SUMMARY OF THE INVENTION

The inventors have now identified a region of AAV-2 genome that behaves like a replication origin in the presence of Rep proteins and adenovirus. All over this application, this region will be referred to as “cis-acting replication element”, or “CARE”. The inventors have then shown that various factors could induce the replication of a sequence functionally linked to a CARE in the presence of Rep proteins. These factors will be designated as “CARE-dependent replication inducers”, or “CARE-DRI”.

The invention described in the present application is an important breakthrough in rAAV production. Indeed, the identification of factors implicated in wild-type AAV replication and production in natural infections allows the design of packaging cell-lines and vectors that will mimic the natural infection process, resulting in better qualitative and quantitative vector production.

As described in Example 1, the integrated rep-cap copies present in HeRC32 cells undergo a dramatic amplification following infection by adenovirus, despite the fact that these rep-cap sequences are devoid of AAV ITRs. The inventors have shown that the amplified rep-cap sequences were extra-chromosomal (Example 2), and that amplification was performed by cellular polymerases rather than adenovirus polymerase (Example 3).

Most importantly, a cis-acting replication element (CARE) was found to be comprised between nucleotides (nt) 190 and 540 of AAV-2 genome (Example 7), more precisely between nt 190 and 361 (Example 12). As evidenced in Example 8, this CARE sequence behaves in vitro and in vivo as a Rep-dependent origin of replication, and is most probably responsible for the dramatic rep-cap amplification observed in Examples 1 and 2.

Therefore, the invention pertains to improved rAAV packaging cell-lines in which transcomplementing AAV genes are operably linked to a CARE sequence.

The invention also pertains to methods of producing recombinant AAV preparations, using a packaging cell-line of the invention, and comprising a step of contacting said rAAV-packaging cell-line with a potent CARE-dependent replication inducer (CARE-DRI).

More generally, the present application describes a CARE/Rep/CARE-DRI system, which enables the amplification of a DNA sequence operably linked to a CARE in the presence of a CARE-DRI and Rep proteins (Rep68 is sufficient, as shown in Example 8).

### DETAILED DESCRIPTION

Throughout this application, several words are employed, the meaning of which should be understood according to the following definitions:

A sequence S2 is “derived from” a sequence S1 if S2 is a fragment of S1, or a variant of S1, or a variant of a fragment of S1. A sequence comprising S1 or a fragment of S1, or a variant of S1, or a variant of a fragment of S1 is said “derived from” S1 as well. In this definition, a “fragment” is longer than 10 nucleotides. In the whole application, a “variant” of a nucleotide sequence designates a sequence which is at least 90% identical to the reference polynucleotide, the percentage of nucleic acid identity between two nucleic acid sequences being calculated using the BLAST software (Version 2.06 of September 1998).

A virus V2 is “derived from” a virus V1 if its genome is derived from that of V1 according to the above definition.

A stable cell-line C2 will be said “derived from” a cell-line C1 if C2 has been obtained by sub-cloning C1 cells, optionally after introducing a foreign DNA into C1 cells.

A cis-acting replication element (CARE), is a nucleotide sequence derived from the sequence from nucleotide position 190 to nucleotide position 540 of the wild-type AAV-2 genome, that promotes the replication of a nucleotide sequence to which it is operably linked, in the presence of Rep proteins (for example, Rep68) and a CARE-dependent replication inducer (CARE-DRI). This replication can be observed either in vitro, as shown in Example 8, or in vivo, for example in HeLa-derived cells, as described at least in Examples 4, 6, 7, 8, 9, and 11. A sequence “operably linked” to a CARE is part of the same nucleic acid molecule as the CARE.

The orientation of a CARE (sense or antisense) is determined according to its natural environment, i.e., the AAV genome or any sequence derived from said genome.

A CARE-dependent replication inducer (CARE-DRI) is a factor able to promote the replication of a sequence operably linked to a CARE, in the presence of Rep proteins. A reference assay to determine whether a candidate is a CARE-DRI consists in contacting HeRC32 cells (described in [7]) with said candidate, in conditions enabling the candidate to penetrate into the cells, and measuring the rep-cap copy number, as described in Example 1. The first identified CARE-DRI was the Adenovirus. The inventors have then identified the adenoviral DNA-binding protein (Ad DBP), as responsible for CARE-dependent replication induction. Herpesvirus is also a potent CARE-DRI. As Herpesvirus does not express the Ad DBP, one or several proteins from this virus might be identified later as CARE-DRIs.

In this application, the words “Adenovirus” and “Herpesvirus” refer to any wild-type virus of the Adenoviridae and Herpesviridae families, respectively, as defined in Virology, Ed. B. N. Fields. New York, Raven Press. In the present application, “Adenovirus” and “Herpesvirus” also refer to any natural mutant of said viruses, as well as to any recombinant virus derived from said viruses.

This application pertains to an isolated nucleic acid sequence comprising a first DNA sequence comprising a cis-acting replication element (CARE) from an Adeno-Associated Virus (AAV), and a second DNA sequence operably linked to said CARE, wherein amplification of said isolated nucleic acid sequence occurs when said isolated nucleic acid sequence is introduced in a cell and said cell is contacted with a CARE-dependent replication inducer (CARE-DRI).

The nucleotide sequence of the CARE of this invention corresponds to the sequence of AAV-2 genome from nucleotide position 190 to nucleotide position 540, or to any fragment of said sequence, or to a variant of said sequence or fragment thereof, provided said fragment or variant still promotes the amplification of a DNA sequence integrated into the genome of a cell and operably linked to said fragment or variant, following contacting said cell with a CARE-DRI.

The isolated nucleic acid according to the invention can comprise a CARE and a polynucleotide sequence heterologous to AAV. In this case, the polynucleotide sequence heterologous to AAV can comprise a polylinker, comprising several cloning sites.

The isolated nucleic acid according to the invention can further comprise genetic elements from a virus, such as retroviral Long Terminal Repeats (LTRs), for example,

Also enclosed in the scope of the invention is a highly producing rAAV packaging cell-line comprising:

an integrated copy of the rep and cap genes, operably linked to an intact or reduced CARE region; and

an integrated copy of an AAV-derived vector, comprising a DNA sequence of interest flanked by AAV Inverted Terminal Repeats (ITRs);

wherein replication of the integrated rep and cap genes is inducible by a CARE-DRI.

In an embodiment of the above-described rAAV packaging cell-line, the integrated AAV-derived vector comprises a CARE sequence, in sense or antisense orientation.

In order to further improve the production rate of a rAAV packaging cell-line, it is possible to integrate an additional cap gene into the genome of said packaging cell-line. This cap gene can be operably linked to a CARE (for instance, a minimal CARE sequence). Indeed, as shown in Example 11, a CARE can promote the replication of a sequence to which it is operably linked, even if this sequence does not contain the rep gene.

Besides the role of CARE in replication, the inventors have demonstrated that the presence of CARE in rAAV vectors increases rAAV titers (illustrated in Example 12). In one embodiment of the packaging cell-lines of the invention, a CARE sequence is inserted into the genome of a vector. In order to prevent the formation of replicative rAAV particles through homologous recombination between the CARE linked to transcomplementing genes and that inserted into the vector genome, the CARE sequences can be linked to the transcomplementing genes in sense orientation, and inserted in the vector in antisense orientation, as described in Examples 14 and 16. Of course, it is also possible to design packaging cell-lines of the invention differently, for example by inserting a CARE in sense orientation in a vector genome and linking a CARE in antisense orientation to transcomplementing genes. Alternatively, or complementarily, homologous recombination can be hindered by using functional variants of CARE. The functionality of such variants can be assayed for example by cloning said variant into a plasmid, for example the pLZ plasmid described in the “materials and method” section. This plasmid is then transfected into 293 cells, together with the pRep plasmid, and the cells are infected with adenovirus. The functionality of the CARE variant is determined by its ability to promote plasmid replication, tested by Mbo I digestion of total DNA, as described in Example 8.

The invention also pertains to a highly producing rAAV packaging cell-line comprising:

an integrated copy of the rep and cap genes, operably linked to a CARE sequence; and

a second integrated copy of the cap gene, which can be operably linked to a CARE sequence, if necessary.

The integration of one or several DNA constructs (CARE-rep-cap, CARE-cap, vector genome, . . . ) into the genome of a cell, in order to obtain a highly producing packaging cell-line, can be performed by any means known by the skilled artisan. In one embodiment of the invention, retroviral vectors are used to integrate one or several DNA constructs into the genome of rAAV packaging cell-lines. One feature of retrovirus-mediated integration is that the integrated sequence is predictable and easy to control. In that embodiment, one or several of the elements integrated in the cell-lines of the invention is/are flanked by retroviral Long Terminal Repeats (LTRs).

As illustrated in Example 1, the inventors have shown that among the various cell backgrounds examined, rep-cap amplification preferentially occurred in HeLa-derived cell clones. Rep-cap sequences integrated in the genome of 293 and TE671 were barely amplified (FIG. 2). This observation suggests that the HeLa-cell background contains factors enhancing CARE-dependent replication. This characteristic can be related to the presence in these cells of several copies of a E2-deleted HPV18 genome [11]. Indeed, HPV has been reported to exert a helper activity for AAV replication [12]. In one embodiment of the rAAV packaging cell-lines of the invention, these cells are derived from cell-lines harboring part of the HPV genome, such as HeLa, CaSki or SiHa cells.

A particular packaging cell-line of the invention can be for example derived from HeRC32 cells by the integration of an additional cap gene operably linked to a CARE in sense orientation, and further carrying one or more integrated copies of a vector genome comprising a CARE in antisense orientation between the AAV ITRs.

The observed properties of HeLa-derived cell-lines can also be related to the presence of a cell-type specific factor, different from HPV sequences, which might be present in other cells. rAAV packaging cell-lines of the invention are therefore in no way limited to cells harboring part of the HPV genome.

An important feature of the packaging cells of the invention is the presence of one or several CAREs integrated in their genome and enabling a significant replication of transcomplementing sequences in the presence of Rep proteins and a so-called CARE-dependent replication inducer (CARE-DRI). As explained above, the first identified CARE-DRI was adenovirus. The inventors have now precisely identified which part of the adenovirus is responsible for CARE-dependent replication induction. Indeed, as evidenced in Example 4, the adenoviral DNA Binding Protein (Ad DBP, or DBP), is necessary and sufficient to promote rep-cap amplification in HeRC32 cells. The DBP is therefore a CARE-DRI per se, which does not exclude the possibility that other adenoviral factors might enhance CARE-dependent replication.

The inventors have then identified another CARE-DRI, which is the Herpesvirus (Examples 9 and 10). Interestingly and unexpectedly, a very strong amplification of the rep-cap signal could be observed following infection of HeRC32 cells with either wild-type herpesvirus or some mutants thereof. This amplification was even stronger in certain conditions to that observed with adenovirus (compare for example lanes 1 and 3 of FIG. 15). Another advantage of herpesvirus as a CARE-DRI is its efficiency in infecting HeRC32 cells. Indeed, virtually 100% of HeRC32 cells can be infected by herpesvirus at a multiplicity of infection (MOI) of 1 pfu/cell, whereas a MOI of 50 to 100 adenoviral pfu/cell is necessary to transduce the same proportion of cells. The results shown in Example 9 further demonstrate that ICP4, ICP27 and ICP0 proteins are not necessary for CARE-dependent replication. One or several herpesviral proteins might be involved in this process.

Another aspect of the invention is a method of producing recombinant AAV preparations, comprising the step of contacting cells harboring rep and cap genes operably linked to a CARE sequence with a CARE-DRI. This method can be performed using a highly producing rAAV packaging cell-line of the invention. Such appropriate rAAV packaging cell-lines have been described above.

In some cases, these cells contain one or several integrated copies of the rAAV genome (illustrated in Examples 14 to 16). rAAV production can then be performed by a process comprising the step of contacting said packaging cells with a helper virus and a CARE-DRI. Of course, the CARE-DRI can be identical to, or part of, the helper virus (for example, when the helper virus is an adenovirus expressing the DBP). Alternatively, the helper functions can be provided by plasmid transfection.

A method of producing recombinant AAV preparations, comprising the step of contacting cells harboring rep and cap genes operably linked to a CARE sequence with a CARE-DRI, is hence part of the present invention. In such a method, the CARE-DRI can be selected from the group comprising Adenoviruses, Herpesviruses, the adenoviral DNA-Binding Protein (Ad DBP), the gene of the Ad DBP, and any gene transfer vector expressing the Ad DBP. In particular methods according to the invention, the CARE-DRI is a herpesvirus mutant from the group comprising ΔICP0, HP66, HR94, and 1178ts, as shown in Example 17.

When the rAAV genome is not present in the packaging cell-line, it can be provided either by DNA transfection, or by infection with a viral vector containing it. In particular, the rAAV genome can be provided by infection by a recombinant helper virus (adenovirus or herpesvirus) carrying said genome.

As illustrated in Examples 10, 15 and 17, the inventors have shown that using herpesvirus as helper can lead to better titers than adenovirus. This correlates with the strong and unexpected CARE-DRI activity of herpesvirus, described in Example 9. Importantly, the inventors have shown that an attenuated mutant like, for example, ΔICP0, HP66, HR94 and 1178ts, can be used efficiently as helper, which had never been described before.

In one embodiment of rAAV production methods of the invention, a packaging cell-line harboring at least one rep-cap copy operably linked to a CARE is infected with a defective herpesvirus carrying a rAAV vector genome.

Another rAAV production method of the invention comprises the step of infecting a packaging cell-line harboring at least one. rep-cap copy operably linked to a CARE in sense orientation, and one rAAV genome comprising a CARE in antisense orientation, with a defective herpesvirus.

A “defective herpesvirus” as mentioned in the two above paragraphs is a herpesvirus that will not undergo a productive infectious cycle in the packaging cell.

The invention also pertains to methods for the selective amplification of a DNA sequence (for example, comprising a gene), in a eukaryotic cell. Such an amplification method comprises for example the steps of (i) linking a CARE to the sequence to be amplified, (ii) introducing said sequence linked to the CARE into a cell, and (iii) contacting said cell with a CARE-DRI. Following step (ii), the sequence operably linked to the CARE can be either extra-chromosomal or Integrated into the cell genome. As the presence of Rep proteins is necessary for CARE-dependent replication, (Rep68 is sufficient, as shown in example 8), these proteins will be provided by any means known by the skilled artisan (for example, transfection of a plasmid encoding rep). For example, the CARE-DRI can be a recombinant adenovirus or herpesvirus encoding rep, or a retrovirus encoding both rep and the Ad DBP. This would simplify the replication induction process, since infection by a single virus would promote the replication of the sequence linked to the CARE. This embodiment of the invention, comprising linking a DNA sequence to be amplified to a CARE, and promoting its replication through the use of Rep proteins and a CARE-DRI, will be referred to later as “CARE/Rep/CARE-DRI system”.

A method of the invention for the amplification of a gene consists in integrating said gene, operably linked to a CARE, into the genome of HeLa cells, and Infecting the resulting cells by a defective herpesvirus carrying the rep gene.

The invention also pertains to a method for the amplification of a DNA sequence operably linked to a CARE and integrated into the genome of a cell, comprising the step of contacting said cell with a CARE-DRI.

In one embodiment of the DNA amplification method described above, the cell-line harbors part of human papilloma virus. For example, this cell-line is selected from the group comprising HeLa, HeRC32, SIHA and CASKI cells, and cells derived thereof.

In the above amplification methods of the invention, the CARE-DRI can be selected from, but is not limited to, the group comprising Adenoviruses, Herpesviruses, the adenoviral DNA-Binding Protein (Ad DBP), the gene of the Ad DBP, and any gene transfer vector expressing the Ad DBP.

The amplification methods of the invention can be used for example for the production of proteins of interest (for example, therapeutic proteins) in eukaryotic cells. Indeed, proteins often undergo post-translational changes in eukaryotic cells, such as glycosylations, which are critical for their biological activity. However, it is difficult to obtain high yields of a recombinant protein in a eukaryotic cell. The use of a CARE/Rep/CARE-DRI system as described above can be a major improvement in recombinant protein production in eukaryotic cells.

In another embodiment of the invention, the CARE/Rep/CARE-DRI system is used in transcomplementing cell-lines for recombinant viruses other than AAV (such as, but not limited to, multidefective adenoviruses). Indeed, the production of defective recombinant viruses, in particular gene transfer vectors for gene therapy, requires transcomplementation for the viral functions deleted from the vector genome. These functions can be provided by the use of a helper virus or by integration of the deleted gene(s) into the genome of transcomplementing cells. However, it is difficult to obtain an efficient transcomplementation for viral proteins (in particular, for capsid proteins) from only a few integrated copies of the corresponding gene(s). The use of the CARE/Rep/CARE-DRI system as described above can lead to an inducible and strong amplification of transcomplementing gene(s), enabling efficient production of (multi)defective viral vectors.

The CARE/Rep/CARE-DRI system can also be used for inducibly over-express a gene within a transgenic animal, for example in order to study the function of said gene. To that purpose, a transgenic animal, bearing the gene to be studied linked to a CARE, must be obtained. This gene can be for example put under the control of the AAV p5 promoter. Local infection with a viral vector expressing both the rep gene and a CARE-DRI will then lead to a spatio-temporal induction of the gene under study.

The invention lastly pertains to a kit for amplifying a DNA sequence in a cell, comprising (i) a nucleic acid comprising a first DNA sequence comprising a cis-acting replication element (CARE) from an Adeno-Associated Virus (AAV), and a second DNA sequence operably linked to said CARE, and (ii) a CARE-DRI, which is selected for example from the group comprising Adenoviruses, Herpesviruses, the adenoviral DNA-Binding Protein (Ad DBP), the gene of the Ad DBP, and any gene transfer vector expressing the Ad DBP, and (iii) expression means to express Rep proteins (Rep 68 is sufficient). To obtain Rep proteins, the skilled artisan can use any expression means known in the art, such as plasmids or recombinant viral vectors derived for example from Adenoviruses, Retroviruses or Herpesviruses. The CARE-DRI (ii) and the Rep expression means (iii) can be joined, for example in the case of a recombinant adenovirus or herpesvirus encoding rep. Alternatively, Rep proteins can be provided as purified proteins.

The hereinafter examples and drawings illustrate the invention without limitating the scope thereof.

### BRIEF DESCRIPTION OF THE DRAWINGS

FIG. 1. Kinetics of rep-cap amplification upon adenovirus infection. HeRC32 cells were infected with Ad5 at a multiplicity of infection (MOI) of 50. Total genomic DNA extracted at 24, 48, and 72 hrs post-infection, was digested with Pst I and analyzed on a Southern blot using a rep probe (1.4 kb) obtained by digesting plasmid pspRC with Pst I. The position of the expected 1.4 kb rep band is indicated. The standard samples with 1, 10, and 100 rep-cap copies per cell were obtained by adding 36, 360, and 3600 pg, respectively, of plasmid pspRC to 10 μg of total genomic DNA from non-Infected HeLa cells. Lane 1: DNA from adenovirus-infected HeLa cells; lanes 2, 3 and 4: standard rep-cap genome copies; lane 5: DNA from non-infected HeRC32 cells; lanes 6, 7 and 8: DNA extracted from HeRC32 cells 24, 48, and 72 h post-adenovirus infection, respectively.

FIG. 2. Analysis of rep-cap amplification in different stable rep-cap cell clones. The stable rep-cap cell clones analyzed are: HeRC32, B50 (derived from HeLa cells, (13)), 293RC21 (derived from 293 cells) and TERC21 (derived from TE671 cells). Rep-cap amplification was analyzed as described in the legend of FIG. 1 following adenovirus infection of the cells at a MOI of 50 (for HeLa-derived cells), 10 (for 293-derived cells), and 25 (for TE671-derived cells). Lanes 1 and 2: standard rep-cap genome copies; lane 3: DNA from adenovirus-infected HeLa cells; lane 4, 6, 8, and 10: DNA from non-infected HeRC32, B50, 293RC21, and TERC21 cells, respectively; lanes 5, 7, 9, and 11: DNA from adenovirus-infected HeRC32, B50, 293RC21, and TERC21 cells, respectively.

FIG. 3. FISH analysis of non-infected and adenovirus-infected HeRC32 and B50 cells. Cells were prepared for FISH analysis as described in the Materials and Methods, and analyzed using a fluorescein-labeled rep-cap probe (4.5 kb) obtained by digesting pspRC with Xba I. Panel A: non-infected HeRC32 cells; panel B: adenovirus infected HeRC32 cells (MOI of 50); panel C: adenovirus-infected HeRC32 cells (MOI of 1); panel D: non-infected B50 cells; panel E: adenovirus-infected B50 cells (MOI of 50); panel F: non infected control HeLa cells. Magnification ×1000.

FIG. 4. Analysis of rep-cap amplified DNA molecules by pulsed-field gel electrophoresis. Samples for pulsed-field gel electrophoresis were prepared from non-infected or adenovirus-infected HeRC32 cells (MOI of 50) as described in the Materials and Methods section and analyzed using a rep probe (1.4 kb). Where indicated, DNA was digested with Not I, which does not cut in the rep-cap genome. A. lanes 1 and 2: non-infected HeLa and HeRC32 cells, respectively; lanes 3 and 4; adenovirus-infected (48 h) HeLa and HeRC32 cells, respectively. B. Lanes 1 and 2: non-infected HeRC32 cells; lanes 3 and 4: adenovirus-infected (48 h) HeRC32 cells. The two arrows indicate the position of the integrated (a) and extra-chromosomal (b) rep-cap fragments.

FIG. 5. A. Effect of adenovirus thermosensitive mutants on rep-cap amplification. HeRC32 cells were infected with Ad.ts125 or Ad.ts149 at an MOI of 50 and incubated at either 32° C. or 39° C. Forty-eight hours later, total genomic DNA was extracted and analyzed using a rep probe as indicated in the legend of FIG. 1. Lanes 1 and 2: standard rep-cap genome copies; lane 3: DNA from non-infected HeRC32 cells; lanes 4 and 5: DNA from HeRC32 cells infected with Ad.ts125 at 32 and 39° C., respectively; lanes 6 and 7: DNA from HeRC32 cells infected with Ad.ts149 at 32 and 39° C., respectively. The position of the expected 1.4 kb rep band is indicated. B. Effect of aphidicolin on adenovirus-induced rep-cap amplification. HeRC32 cells infected with Ad5 (MOI of 50) for 2 h at 37° C. and then either left untreated or incubated in the presence of aphidicolin at the indicated final concentrations. Two μg of total DNA extracted 48 h later was analyzed by dot blot using a rep (1.4 kb) or DBP (1.6 kb) probe. The DBP probe was obtained by digesting the pMSG-DBP-EN plasmid (19) with Hind III and Sfi I. Lane 1: DNA from non-infected HeRC32 cells; lane 2: DNA from adenovirus-infected HeRC32 cells; lanes 3 to 6: DNA from adenovirus-infected HeRC32 cells incubated in the presence of increasing concentrations of aphidicolin.

FIG. 6. A. Effect of the adenovirus DBP on rep-cap amplification. HeRC32 cells were infected with Ad.ts125 (MOI of 50) at the indicated temperature and total DNA analyzed by Southern blot using a rep probe (1.4 kb) as indicated in the legend of FIG. 1. Where indicated, the CMVDBP plasmid (10 μg) was transfected into 4×10⁶ HeRC32 cells using Exgen (EuroMedex), either alone or 6 h prior adenovirus infection. In this case, the transfection was done at 37° C. and the cells were switched to the indicated temperature immediately after adenoviral infection. Lane 1: DNA from non-infected HeLa cells; lanes 2, 3 and 4: standard rep-cap genome copies; lane 5: DNA from HeRC32 cells infected with Ad.ts125 at 32° C.; lane 6: DNA from HeRC32 cells transfected with CMVDBP and Infected with Ad.ts125 at 32° C.; lane 7; DNA from HeRC32 cells infected with Ad.ts125 at 39° C.; lane 8: DNA from HeRC32 cells transfected with CMVDBP and infected with Ad.ts125 at 39° C.; Lane 9: DNA from non-infected HeRC32 cells; lane 10: DNA from HeRC32 cells transfected with the CMVDBP plasmid. B. Analysis of rep-cap amplification in ΔRep-HeLa cells. Total DNA was extracted from uninfected (lane 1) and adenovirus-infected (lane 2) ΔRep-Hela cells, digested with Pst I and analyzed on a Southern blot as previously indicated. Since the deletion in the rep sequence removes one Pst I site, the expected band is 3.8 Kb in size.

FIG. 7. FISH analysis of HeRC32 cells transfected with the CMVDBP plasmid. 4×10⁶ HeRC32 cells were transfected with 10 μg of the CMVDBP plasmid using Exgen (EuroMedex). Forty-eight hours later, the cells were prepared for FISH analysis as indicated in the Materials and Methods. The samples were analyzed using a fluorescein-labeled rep-cap probe. Two typical examples of rep-cap amplification are shown. Panel A: untransfected HeRC32 cells; panels B and C: transfected HeRC32 cells. Magnification ×1000.

FIG. 8. Detection of Rep and DBP proteins following transfection of the CMVDBP plasmid into HeRC32 cells. 6×10⁴ HeRC32 cells grown on glass slides were transfected with 0.4 μg of the CMV.DBP plasmid. Forty-eight hours later, the cells were fixed and analyzed by immunofluorescence using an anti-DBP (26) and an anti-Rep 68/40 (panels A, B, and C) or an anti-Rep 78/52 (panels D, E, and F) antibody (39). Cells were photographed with either a fluorescein (panels A and D) or a rhodamine (panels B and E) filter. In panels C and F, the two images are superposed. Magnification ×1000.

FIG. 9. Southern blot analysis of DNA extracted from purified AAV particles. 293 cells were transfected with plasmid pRC either alone (lanes 1, 3, 5, and 7) or in combination with pAAVLZ (lanes 2, 4, 6, and 8) and then infected with adenovirus (Ad.dl324). AAV particles were purified from the cell lysate on a CsCl gradient. DNA, extracted after exhaustive DNase I treatment of the particles, was run on a 1% neutral agarose gel and transferred in neutral conditions to a membrane. The membrane was hybridized to a LacZ (lanes land2), REP (lanes 3 and 4), CAP (lanes 5 and 6), or AAV ITR (lanes 7 and 8) probe.

FIG. 10. In vivo replication analysis of plasmid pRCtag. A. Circular map of pRCtag plasmid: the tag sequence located at the 3′ end of the rep-cap genes is represented by the hatched area. The position of the two relevant Dpn I/Mbo I sites is indicated B. 293 cells were transfected with the pRCtag plasmid and subsequently infected (+Ad: lanes 7, 8, and 9) or not (−Ad: lanes 4, 5, and 6) with adenovirus. Total DNA was extracted 48 hrs later, and digested with Dpn I or Mbo 1. The samples were then run on a 1% agarose gel, transferred to a membrane and hybridized to a tag probe. As a control (C: lanes 1, 2, and 3), untransfected pRCtag plasmid DNA mixed with 2 μg of total DNA from 293 cells, was digested with Dpn I or Mbo I and similarly analyzed using the tag probe. The expected 1430 bp DpnI/Mbo I fragment hybridizing to the tag probe is indicated.

FIG. 11. In vivo replication analysis of plasmid pRCtag/Δ. A. Circular map of the pRCtag/Δ plasmid. This plasmid differs from pRCtag by a 350 bp deletion in the 5′ portion of the rep gene (nt 190-540 of wild type AAV) that removes the entire p5 promoter and the 5′ portion of the rep ORF. The position of the two relevant Dpn I/Mbo I sites is indicated. B. 293 cells were transfected with the pRCtag/Δ (lanes 4 to 9) or the pRCtag (lanes 10 to 12) plasmid, in the presence (+pRep) or in the absence (−pRep) of a plasmid encoding for the four Rep proteins under the control of the AAV p5 and p19 promoters. pRCtag/Δ was similarly transfected into HeRC32 cells (lanes 13 to 15) which harbor one integrated copy of the ITR-deleted AAV-2 genome (4). Cells were subsequently infected with adenovirus (+Ad). Total DNA was extracted 48 hrs later and analyzed as described in the legend of FIG. 10. Untransfected pRCtag/Δ plasmid DNA (C: lanes 1,2, and 3), mixed with 2 μg of total DNA from 293 cells, was used as a control for Dpn I and MboI digestion.

FIG. 12. Southern blot analysis of DNA extracted, from purified AAV particles upon transfection by pRCtag and pRCtag/Δ. HeRC32 cells were transfected either with pRCtag (lane 1) or pRCtag/Δ (lane 2) and then infected with adenovirus. DNA extracted from the purified AAV particles after exhaustive DNase I treatment was analyzed in a Southern blot experiment, using a tag probe as previously described.

FIG. 13. In vivo replication assay of the pLZCARE plasmids. A. Circular map of pLZCARE plasmids. The CARE sequence (190 to 540 bp of wild type AAV) indicated by the hatched area was introduced upstream of the CMV LacZ cassette either in the same (pLZCARE+) or In the opposite (pLZCARE−) orientation. The position of the relevant Dpn I/Mbo I sites is indicated on the map, B. 293 cells were transfected with the pLacZ (lanes 1 to 3), the pLZCARE+ (lanes 7 to 18) or the pLZCARE− (lanes 19 to 21) plasmid, in the presence (+pRep), or in the absence (pRep), of the pRep plasmid and subsequently infected (+Ad) or not (−Ad) by adenovirus. Total DNA was extracted 48 hrs later and analyzed as described in the legend of FIG. 10 using a LacZ probe. Untransfected pLZCARE+ plasmid, mixed with 2 μg of total DNA from 293 cells, was used as control for Dpn I and Mbo I digestion (C: lanes 4, 5, and 6).

FIG. 14. In vitro replication assay of the pLZCARE plasmids. A. Circular map of the pLZ and pLZCARE+/−plasmids. Two major linear species are generated upon EcoR I digestion: one of 3077 bp that is common to both plasmids and corresponds to the LacZ gene, and one of 3261 and 3690 bp for pLacZ and pLZCARE+/−, respectively, that corresponds to the CARE sequence associated with the rest of the plasmid. B. The EcoR I-digested plasmid DNA was used directly in the in vitro replication assay, so each reaction contains equimolar amounts of the two larger DNA fragments. The experimental conditions are described in the Materials and Methods section, in the “Examples” part of the application. Replication assays were performed using a cellular extract from uninfected HeLa cells, supplemented (lanes 2, 4, and 6) or not (lanes 1, 3, and 5) with purified Rep68 protein.

FIG. 15. rep-cap signal amplification observed after wild-type HSV-1 infection. HeRC32 cells were infected with wild-type HSV-1 and harvested 48 to 72 hrs post-infection. The number of rep-cap copies was then estimated by Southern blot, using a rep-cap probe,

Lane 1: HeRC32 cells wild-type adenovirus (MOI=50). Lane 2: uninfected HeRC32 cells. Lanes 3 to 7: HeRC32 cells+HSV-1 (MOIs of 20, 10, 5, 1 and 0.5 pfu/cell, respectively). Lanes 8 and 9: 100 and 10 psp RC plasmid copies per cell, respectively (rep-cap scale). Lane 10: uninfected HeLa cells.

FIG. 16. LacZ gene amplification in two CARE-LacZ clones HeLa-CARE-LacZ cells were transfected with plasmid Rep.pA and infected by wild-type adenovirus. The cells were harvested 48 hrs post infection, total genomic DNA was extracted and analyzed by Southern blot using a LacZ probe. Lane 1: 50 CARE-lacZ plasmid copies per cell. Lane 2: size ladder. Lanes 3 and 7: CARE-LacZ clones (n° 2 and 14, respectively), untransfected and uninfected. Lanes 4 and 8: clones 2 and 14, respectively, infected by wild-type adenovirus. Lanes 5 and 9: clones 2 and 14, respectively, transfected by plasmid Rep-pA. Lanes 6 and 10: clones 2 and 14, respectively, transfected with plasmid Rep.pA and infected by wild-type adenovirus.

FIG. 17. Structure of rAAV vectors containing a CARE sequence. The vectors were obtained as detailed in the Materials and Methods section. The three vectors presented contain the nlsLacZ gene placed under the control of the CMV promoter. The CARE sequence was inserted between the 5′ ITR and the CMV promoter in either the sense (pAAVLZ/CARE+) or the antisense (pAAVLZ/CARE−) orientation. A control vector (pAAVLZ/C) was obtained by inserting, at the same position as CARE, an unrelated sequence derived from the human BGT-1 cDNA. As a result, the size of the vectors (ITR to ITR) is 4810 bp for the AAVLZ/CARE+/− and 4860 bp for the AAVLZ/C vector.

FIG. 18. Comparison of rAAV yields obtained in the presence or absence of CARE. HeRC32 cells were transfected with pAAVLZ/C, pAAVLZ/CARE+, or pAAVLZ/CARE− and subsequently infected with adenovirus. rAAV preparations 1 and 2 were obtained from 6×15-cm plates of cells, whereas preparations 3 and 4 were obtained from 2×15-cm plates of cells. rAAV in each preparation was titrated either after purification on a CsCl gradient (rAAV # 2, 3, and 4) or directly in the crude cell lysate (rAAV # 1). For each preparation, we measured: A) the number of genome-containing particles by dot blot; B) the number of infectious particles by mRCA [10]; and C) the number of transducing particles using an LFU assay. The titers obtained for the control rAAVLZ/C stock were arbitrarily set to one. The presence of contaminating rep-positive particles as detected by mRCA is indicated by (*).

FIG. 19. Titration by mRCA of the number of infectious particles produced using the pAAVLZ/C, the pAAVLZ/CARE+, or the pAAVLZ/CARE− plasmid. A typical example (preparation 3) of the titration result obtained by mRCA is shown. The assay was performed as described [10] using adenovirus-infected HeRC32 cells. Each dot visualizes an infectious particle able to replicate under these conditions, and to generate several copies of rAAV genomes hybridizing to the LacZ probe.

FIG. 20. Production of AAVGFP. AAVGFP was produced in standard conditions (cotransfection of 293 cells by pDG and PAAVGFP) and in HeRC32/AAVCAREeGFP cells infected with Adenovirus (MOI=50) or Herpesvirus (MOI=1). The cells were collected 48 hrs post infection by the Adenovirus and 24 hrs post infection by the Herpesvirus, and the resulting preparations were titrated by dot blot as explained below. Each column corresponds to the result (in particles/cell) of one independent experiment.

FIG. 21. Production of AAVGFP. The same preparations as described in FIG. 20 were titrated by modified RCA as explained below. Each column corresponds to the result (in infectious particles/cell) of one independent experiment.

FIG. 22. Comparison of different mutants of HSV for CARE-dependent replication induction. HeRC32/AAVCAREeGFP cells were infected with Ad5 (MOI=50), HSV-1, ΔICP1, ΔICP4, ΔICP27, HP66, HR94, 1178ts (MOI=1), collected 24 hrs post infection by HSV-1 and HSV mutants and 48 hrs post infection by Ad5. The titers of the obtained rAAV preparations (in infectious particles) were measured by a modified RCA assay.

### EXAMPLES

The following examples can be performed using the materials and methods described below:

Materials and Methods

DNA Constructs.

Rep-cap plasmids. Plasmid pspRC [7] contained the ITR-deleted AAV genome (nt 190 to 4484 of wild type AAV) and was obtained by excising the rep-cap fragment from plasmid psub201 by Xba I digestion [13] and by inserting it in the Xba I site of plasmid pSP72 (Promega). The pRC plasmid contained the same ITR-deleted AAV-2 genome excised as a Xba I fragment from psub201, and inserted into the Xba I site of pBluescript SK+ (Stratagene). The pRCtag/Δ plasmid contains a 350 bp deleted rep-cap sequence (nt 191 to 540 of the wild type AAV) followed at the 3′end of the AAV sequences by 404 bp from φX174 DNA. The pRCtag plasmid was obtained by inserting a 404 bp fragment from ‘PX174 DNA, in pRC at the 3’ end of the rep-cap sequence. The pRCtag/Δ plasmid was derived from pRCtag by removing 350 bp located at the 5′ end of the rep-cap genome (nt 191 to 540 of wild type AAV). The dITR-RC plasmid contained the ITR-deleted rep-cap genome inserted between the adenovirus ITRs. The pRep plasmid contains the rep genes under the control of the AAV p5 and p19 promoters followed by the bovine growth hormone pA signal, inserted in the pSP72 backbone.

CMVDBP. To obtain the CMVDBP construct, the pMSG-DBP-EN plasmid [14] was digested with Kpn I, filled in with T4 polymerase, and subsequently digested with Hind III. The resulting band containing the E2a gene was gel-purified and inserted into the blunt-ended pRC/CMV plasmid (Promega) which had been digested with Hind III and Xba I.

pLZ and pLZCARE plasmids: The CMV immediate early promoter and the bovine growth hormone polyadenylation signal (BGH pA) were excised from the pRC/CMV plasmid (Invitrogen) with Nde I and Pvu II. This fragment was blunted with Klenow enzyme and ligated between the EcoR V-Pvu II sites of pSP72 (Promega) to give plasmid pCMV.pA. To obtain pLZ, a 3,550 bp LacZ gene linked to a nuclear localization signal (nls), was inserted in the BamH I site of pCMV.pA. The CARE sequence corresponding to nt 191 to 540 of wild type AAV was excised from pspRC with BgI II-Sfi I. A 24 bp double-stranded oligonucleotide (5′ GATCTCTAGTCAGTTAGGCCTCCG 3′) was ligated at the Sfi I site to introduce a stop codon, in each possible open reading frame, and a BgI II site at the 3′ end of CARE. The resulting construct was then cloned into the BgI II site of the pLZ plasmid to give constructs pLZCARE+ and pLZCARE−, in which CARE was cloned upstream the CMVLacZ cassette in sense or antisense orientation, respectively.

pAAVLZ, pAAVLZ/CARE, and pAAVLZ/C vector plasmids: pAAVLZ was derived from SSV9 [13] by removing the rep-cap sequence with SnaB I and replacing it with the CMVnlsLacZ cassette. To generate the pAAVLZ/CARE plasmids, the CARE sequence was cloned either in the sense (pAAVLZ/CARE+) or antisense (pAAVLZ/CARE−) orientation between the 5′AAV ITR and the CMV promoter. For the control plasmid (pAAVLZ/C), an irrelevant 380 bp sequence from the human bilirubine glycuronyl transferase-1 (BGT-1) cDNA was cloned in place of CARE. Cell lines and viruses

293, 293RC21, TERC21, HeLa, HeRC32 and HeRC32/AAVCAREeGFP cells were maintained in Dulbecco's modified Eagle's medium (DMEM, SIGMA), supplemented with 10% heat-inactivated fetal bovine serum (FBS) (HyClone) and 1% (vol/vol) penicillin/streptomycin (GIBCO BRL, 5,000 U/ml). HeRC32, 293RC21, TERC21 cell clones were obtained by co-transfecting plasmid pspRC which harbors the ITR-deleted rep-cap genome (bp 190 to 4484 of wild type AAV) with plasmid PGK-Neo, conferring resistance to G418 into HeLa, 293 and TE671 (a human medulloblastoma cell line) cells, respectively. The ΔRep-HeLa cell clone was obtained using the pRCtag/Δ plasmid in which 350 bp located at the 5′ end of the rep-cap genome (corresponding to nt 191 to 540 of the wild type AAV) were deleted. The isolation and characterization of HeRC32 and 293RC21 cells have been [7]. TERC21 and ΔRep-HeLa cells were similarly characterized and shown to have one or less integrated rep-cap copy per cell genome. The B50 cell line, kindly provided by J. Wilson (U. Penn), is a HeLa derived cell clone harboring a stably integrated ITR-deleted rep-cap genome [5]. The adenoviruses used were: wild type adenovirus type 5 (Ad5) (ATCC VR-5), two thermosensitive strains having a mutation in the E2a (Ad.ts125) and the E2b gene (Ad.ts149) [15], and ΔE1 Ad.dl324 (a gift from Transgène, France). Adenoviruses were produced and titrated on 293 cells using standard [16]. Herpesviruses were titrated according to the procedures described by Timbury et al. [17]. The absence of revertants in the purified stock of Ad.ts125 and Ad.ts149 was tested at non permissive temperature. The absence of contaminating wild type AAV in the three parental cell lines (HeLa, 293, and TE671) and the adenoviral stocks was determined by PCR analysis using rep primers [10].

Analysis of Total Genomic DNA by Southern Blot

Total DNA was extracted by lysing the cells in a 10 mM Tris-HCl pH 7.5/1 mM EDTA/100 mM NaCl/1% SDS solution containing 500 μg/ml of proteinase K (Boehringer Manheim). After overnight digestion at 50° C., the DNA was extracted twice with phenol/chloroform and precipitated.

For analysis, DNA was digested with the enzyme indicated, run on a 1% agarose gel, and transferred under alkaline conditions (NaOH 0.4 N) to a Hybond N⁺ membrane (Amersham). The membrane was hybridized to a fluorescein-labelled probe (Amersham, Gene Images random prime labelling module) and incubated overnight at 65° C. The following day the membrane was washed in 1×SSC (Research Organics)/0.1% SDS and then in 0.1×SSC/0.1% SDS, for 15 min at 65° C. each. The membrane was then processed according to the manufacturer's protocol (Amersham, Gene Images CDP-star detection module) and exposed to autoradiography film.

Analysis of Total Genomic DNA Pulsed-Field Gel Electrophoresis

Cells were harvested by trypsinization, washed with phosphate-buffered saline (PBS) (KCl 2.5 mM, KH₂PO₄ 1.5 mM, NaCl 137 mM, Na₂HPO₄ 8 mM, pH 7.4) at 37° C., resuspended at 4×10⁷ cells/ml, and gently mixed with an equal volume of a 1% solution of low-melting agarose (Seaplaque, FMC Bioproducts) in Mg²⁺, Ca²⁺-free PBS precooled at 50° C. The mixture was allowed to solidify in the cold and agarose-cell plugs were then treated with proteinase K (2 mg/ml) in the presence of 1% SDS. After washing, the plugs were stored at 4° C. in 20 mM Tris buffer, 5 mM EDTA, pH 8.0. For digestion, the plugs were incubated with 50 U of enzyme in a total volume of 300 μl per plug and incubated for 6 h at 37° C. Electrophoresis was carried out using 1% agarose gels (SeaKem ME agarose, from FMC Bioproducts in 0.5×TBE buffer (TBE: 90 mM Tris, 90 mM borate, 2 mM EDTA, pH 8.0) at 6V/cm for 14-20 h with a switching time of 50-90 s, using recirculating 0.5×TBE. After EtBr staining and UV visualization, the DNA was transferred on a Hybond-N⁺ membrane under alkaline conditions (NaOH 0.4 N). The membrane was treated and hybridized as described above.

Immunofluorescence Analysis

Immunofluorescence analysis was performed on 5×10⁴ cells seeded on glass slides. After washing for 5 min in PBS, the cells were fixed in 4% paraformaldehyde in PBS for 20 min at room temperature and then permeabilized with 2% Triton X-100 in PBS for 20 min at room temperature (RT). After washing in PBS, the cells were incubated with 2% BSA in PBS for 20 min at RT and then incubated with the appropriate antibody. The primary antibody was diluted in PBS/0.1% Tween and incubated for 1 h with the fixed cells at RT. The monoclonal anti-DBP mouse antibody (kindly provided by A. Levine, [18]) was diluted 1/10, the polyclonal anti-Rep guinea pig antibodies (kindly provided by J. Kleinschmidt, [19]) were diluted 1/100. Next, the slides were washed in PBS and then incubated with a fluoresceinated anti-mouse antibody (Amersham) and a rhodamine anti-guinea-pig antibody diluted 1/200 and 1/50, respectively, in PBS/0.1% Tween for 1 hr at RT in the dark. After washing in PBS, the cells were embedded in Vectashield mounting medium (Vector Laboratories, Inc) and analyzed using a confocal LEICA DMiRBE microscope.

Fish Analysis

To obtain metaphase spread, exponentially growing cells were treated with colcemid (40 ng/ml) for 1 h at 37° C. After trypsinization and centrifugation, the cell pellets were resuspended in 75 mM KCl for 35 min at 37° C. After addition of a cold methanol-acetic acid (3:1) solution, cells were pelleted and then resuspended in the same fixative solution for 10 min at 4° C. and finally dropped onto slides. Slides were air-dried and the DNA was denatured in 70% formamide-2×SSC pH 7.0 for 1 min at 75° C. Slides were then dehydrated in an ice-cold ethanol series (70%-85%-100% for 1 min each) and air dried. Hybridization was performed overnight at 37° C. using a fluorescein-labelled probe according to the manufacturers protocol (Nick Translation Reagent Kit, Vysis Inc). Slides were then washed sequentially in 2×SSC for 2 min at 75° C. and in 2×SSC-0.1% Triton for 2 min at RT. After air drying in the dark, slides were dehydrated and mounted with an anti-fade DAPI solution. Hybridization signals were visualized by using a Zeiss Axioplan 2 fluorescence microscope with a oil immersion objective.

rAAV Production and Titration.

Recombinant AAV were produced in 293 or HeRC32 cells. In both cases, cells were plated in 15 cm diameter dishes and transfected by the calcium phosphate method at 80% confluence. 293 cells (6×15-cm plates) were co-transfected with the pRC and pAAVLZ plasmids (12.5 μg each per 15-cm plate), whereas HeRC32 cells (2×to 6×15-cm plates) were transfected with pAAVLZ plasmid only (12.5 μg each per 15-cm plate). After six hours, media was replaced by DMEM 5% FBS containing Ad.d1324 at a Multiplicity of Infection (MOI) of 10 for 293 cells, or wild type Ad5 at an MOI of 50 for HeRC32 cells. At the time of the cytopathic effect (48 hrs), cells were harvested, pelleted, resuspended in 10 ml of 10 mM HEPES pH 7.6, 150 mM NaCl buffer, and then lysed by three freeze/thaw cycles. The cell lysate was clarified by centrifugation at 3,000 rpm for 15 min. Where indicated, rAAV was further purified on a cesium chloride gradient as previously described [10] with the difference that rAAV was dialyzed for 1 hr against three changes of Ringer's solution (Baxter) at 4° C. Particles produced using the pRC construct alone, i.e., in the absence of the vector, were similarly processed.

Recombinant AAV preparations were titrated using three different methods: (i) dot blot to determine the DNA-containing particles/ml; (ii) a modified Replication Center Assay (mRCA) to measure the number of infectious particles/ml as well as the contamination with wild type AAV-like particles; and (iii) a LacZ forming unit (LFU) assay to measure the number of transducing rAAV particles/ml. All three methods have been previously [10].

Extraction of Viral DNA and Southern Blot Analysis.

To extract viral DNA, AAV particles (100 μl of the 1.6 ml stock) were first incubated with 50 U of DNase I (Roche) in 500 μl of DMEM for 1 hr at 37° C. Six hundred microliters of 2× proteinase K buffer (20 mMTris-HCl pH 8.0, 20 mMEDTA pH 8.0, 1% SDS) containing 250 μg of proteinase K (Roche) were then added and the reactions incubated for 1 hr at 37° C. After phenol/chloroform extraction, DNA was precipitated in the presence of glycogen, washed in 70% ethanol, and resuspended in water, DNA samples were fractionated on a 1% agarose gel made in Tris-Borate-EDTA (TBE), and transferred onto a Hybond N+ membrane (Amersham Pharmacia Biotech) in neutral conditions (20×SSC), without prior denaturation. Membranes were hybridized with fluorescein-labeled probes (Amersham, Gene Images random prime labeling module) overnight at 65° C. and processed according to the manufacturer's protocol (Amersham, Gene Images CDP-Star detection module) before exposure to autoradiography film. The rep (509 bp) and cap (1410 bp) probes were isolated from pRC plasmid. The AAV (190 bp) and adenovirus (132 bp) ITR probes were obtained from the SSV9 [13] and the dITR.RC plasmid, respectively. The LacZ probe (875 bp) was obtained from the pLZ plasmid and the tag probe (404 bp) was isolated from φX174 DNA.

In Vivo DNA Replication.

293 and HeRC32 cells seeded in a 10-cm plate were transfected at 80% of confluence. 293 cells were transfected using the calcium phosphate method with 5 μg of plasmid DNA. HeRC32 were transfected using Exgen (Euromedex) according to the manufacturer's instructions with 12 μg of plasmid DNA. After 6 hrs, cells were infected with adenovirus (Ad.dl324 for 293 cells at an MOI of 10; wild type Ad5 for HeRC32 at an MOI of 50). At the time of the cytopathic effect, total DNA was extracted by lysing the cells in 10 mM Tris-HCl pH 7.5, 1 mM EDTA, 100 mM NaCl, 1% SDS solution containing 500 μg/ml of Proteinase K (Roche). After overnight incubation at 55° C., DNA was extracted twice with phenol/chloroform and precipitated. Total DNA (1 μg for 293 cells and 5 μg for HeRC32 cells) was digested with Dpn I or MboI for 2×4 hrs at 37° C. Digestion products were separated on 1% agarose gel made in TBE buffer and transferred on a Hybond N+ membrane using denaturing conditions (0.4 N NaOH). Hybridization and detection were performed as described above.

In Vitro (Cell-Free) DNA Replication.

In vitro replication assays were performed as previously described [20]. Briefly, the reaction mixture (15 μl) contained 40 mM HEPES (pH 7.7); 40 mM creatine phosphate (pH 7.7); 7 mM MgCl₂; 4 mM ATP; 200 μM each of CTP, GTP, and UTP, 100 μM each of dATP, dGTP, and dTTP; 10 μM dCTP; 10 μCi of α-³²P-dCTP (3,000 Ci/μmol; Amersham); 2 mM dithiothreitol, 6 mM potassium glutamate; 2.0 μg of creatine phosphokinase; 75 μg of HeLa cell extract protein; 0.1 μg of plasmid DNA, and 100 ng of His-tagged Rep 68. EcoR 1-digested pLZ, pLZCARE+, or pLZCARE-plasmids were used as substrates. Reaction mixtures were preincubated at 37° C. for 3 hrs, at which point Rep 68 and labeled dCTP were added. Incubation was at 37° C. for an additional 16 hrs. The reaction products were brought to 65 μl with digestion buffer (20 nM HEPES pH 7.5, 10 mM KCl, 10 mM EDTA, 1.0% SDS, 50 mM NaCl), passed over a Sephadex-50 spin column, digested with proteinase K (10 mg/ml) for 2 hrs at 50° C., and analyzed by electrophoresis on 0.8% agarose gel in TBE buffer.

### Example 1

### AAV Rep-Cap Gene Amplification is Induced Preferentially in Adenovirus-Infected HeLa-Derived Cell Clones

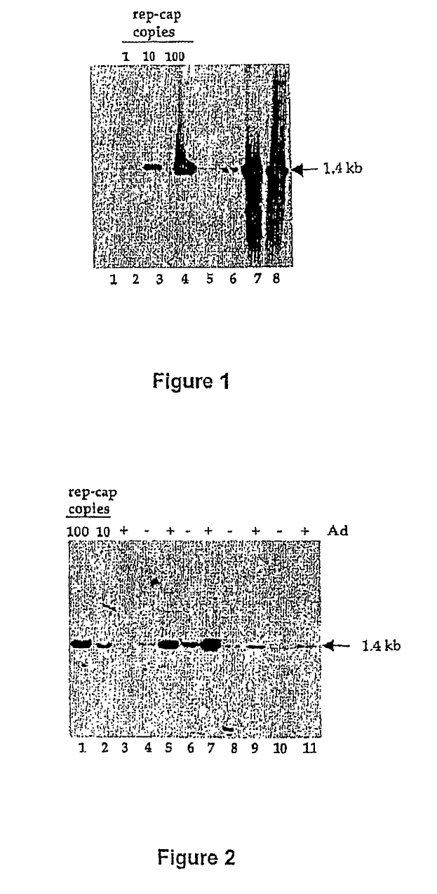

The initial observation underlying this study was made using a Hela-derived cell clone harboring one integrated copy of an ITR-deleted rep-cap genome (HeRC32 cells) [7]. When HeRC32 cells were infected with wild type adenovirus, the integrated rep-cap copies underwent a dramatic amplification leading to a 100-fold increase in the rep-cap copy number, as evidenced by Southern blot analysis of total DNA and hybridization with a rep probe (FIG. 1). The determination of the rep-cap copy number at different time-points indicated that amplification occurred mainly between 24 and 48 hours following adenovirus infection. After the 48 hours time-point no significant increase was detected. To exclude the possibility that this phenomenon was due to an intrinsic property of the HeRC32 cell clone, the same analysis was performed with another Hela-derived rep-cap cell clone (B50), which harbors five integrated rep-cap copies [5]. Despite the different origin of the B50 cells, rep-cap sequences were similarly amplified following adenovirus infection (FIG. 2, lanes 6 and 7). Interestingly, the number of rep-cap copies found in the B50 cells after adenovirus infection was similar to that measured in HeRC32 cells, suggesting that the level of amplification was not dependent upon the initial rep-cap copy number (FIG. 2, compare lanes 5 and 7). The same results were obtained using a cap probe (data not shown), indicating that the entire rep-cap genome had undergone amplification. In addition, other cellular or viral endogenous sequences such as those corresponding to the elongation factor 1-α (EF1-α, the bilirubin glycuronyl transferase-1 (BGT-1), and the human papillomavirus (HPV) genes were not found to be amplified upon adenovirus infection (data not shown), suggesting that the amplification phenomenon was restricted to rep-cap containing sequences.

Further analyses were conducted to determine if rep-cap amplification could also take place in other rep-cap stable cell clones derived from other cell backgrounds. For this, two stable cell clones derived from low passage 293 (293RC21) and TE671 cells (TERC21), harboring integrated rep-cap genomes were similarly analyzed by Southern blot. Following adenovirus infection, the endogenous rep-cap sequences were amplified only two to three-fold in the 293RC21 cells, a level much lower than that observed in HeRC32 and B50 cells (FIG. 2, lanes 8 and 9). In TERC21 cells, no rep-cap amplification was detected (FIG. 2, lanes 10 and 11). Overall, these analysis suggested that adenovirus-induced rep-cap amplification preferentially occurred in the HeLa-derived cell clones analyzed.

### Example 2

### Amplified Rep-Cap Sequences are Extra-Chromosomal

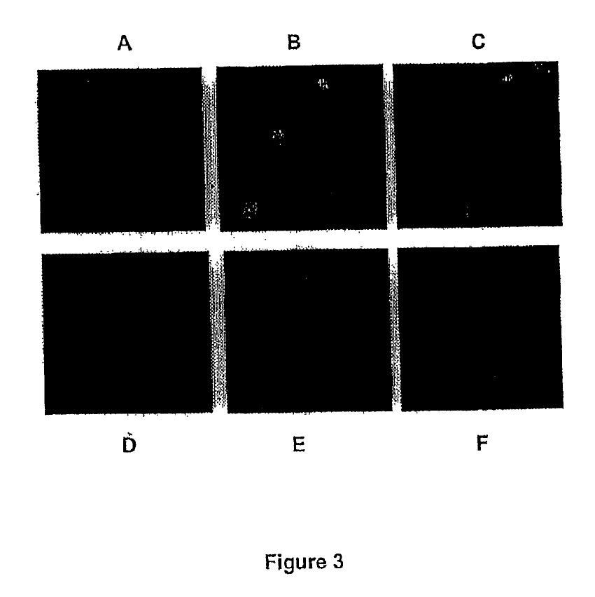

The next question concerned the status of the amplified rep-cap sequences. The inventors wished to determine if the amplified rep-cap sequences are found in an integrated or in an extra-chromosomal form. For this, rep-cap sequences present in control and adenovirus-infected HeRC32 and B50 cells were analyzed by FISH. Metaphase spreads of uninfected cells confirmed the presence of rep-cap sequences in an integrated status in both cell clones (FIG. 3, panels A and D). The analysis performed 48 hours following adenovirus infection showed an increase in the rep-cap signal which appeared as a large dot (FIG. 3, panels B and E). This result illustrated the amplification phenomenon previously detected by Southern blot. However, because of the growth arrest induced by the adenovirus infection, it was not possible to visualize metaphases in these cells and, thus, to distinguish if the rep-cap signal following amplification co-localized with a chromosomal structure. To try to visualize intermediate forms of amplification, HeRC32 cells were infected with wild type adenovirus at a sub-optimal multiplicity of infection (MOI) of 1. In this case, different patterns could be observed. Particularly, some nuclei displayed a strong rep-cap signal, which was not concentrated in a single spot but was rather diffuse (FIG. 3, panel C). This last result suggested that amplified rep-cap sequences were present in an extra-chromosomal form.

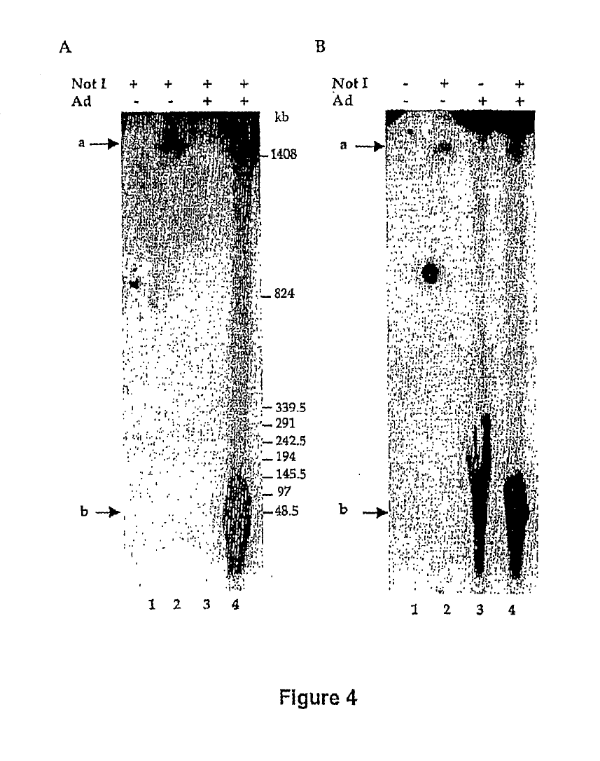

To confirm this observation, total genomic DNA extracted from infected and uninfected HeRC32 cells, was analyzed by pulse-field gel electrophoresis followed by Southern blot analysis using a rep probe. Digestion of total DNA extracted from uninfected HeRC32 cells with Not I, which does not cut the rep-cap DNA, released a unique high molecular weight band presumably containing the integrated rep-cap copies (FIG. 4A, lane 2). Following adenovirus infection of HeRC32 cells, an additional faster-migrating form was detected (FIG. 4A, lane 4). Both of these signals were not detected using DNA from control or adenovirus-infected HeLa cells (FIG. 4A, lanes 1 and 3). The highest molecular weight band seen with DNA from adenovirus-infected HeRC32 cells was not detected using undigested DNA (FIG. 4B, lane 3), highlighting the specificity of the probe. Conversely, the faster-migrating band was still detected using undigested DNA (FIG. 4B, lane 3), suggesting that this form corresponded to an extra-chromosomal molecule containing rep-cap sequences.

### Example 3

### Cellular but not Adenoviral Polymerases are Involved in the Amplification Process

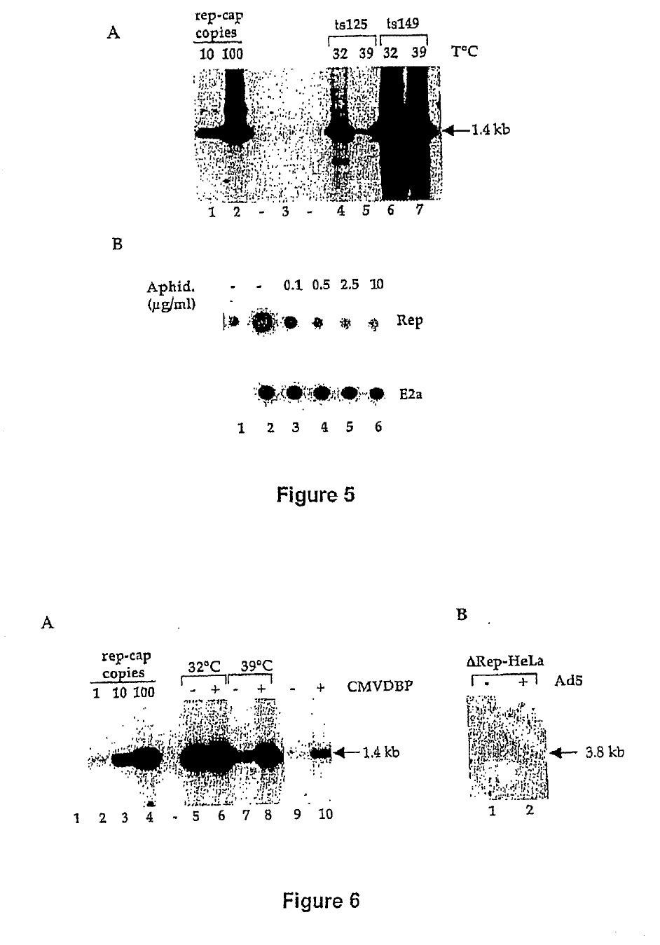

The above results indicated that upon adenovirus infection, integrated rep-cap sequences were amplified and extruded from the chromosomal structure. To further elucidate this phenomenon, it was important to determine if the amplification of rep-cap sequences resulted from the activity of cellular or adenoviral polymerases. To answer this question, rep-cap amplification was analyzed after infection of HeRC32 cells with an adenoviral mutant harboring a thermosensitive mutation in the E2b gene encoding for the viral polymerase (Adts149). HeRC32 cells were infected with Adts149 and maintained for 48 h at either 32° C. (permissive temperature) or 39° C. (non permissive temperature). Analysis of total DNA by Southern blot and hybridization with a rep probe indicated that inactivation of the adenoviral polymerase at 39° C., did not inhibit rep-cap amplification, which reached a level similar to that observed in cells infected at 32° C. (FIG. 5A, lanes 6 and 7). This result indicated that the adenoviral polymerase was not involved in the rep-cap amplification and further suggested the involvement of cellular polymerases in this process.

To confirm this hypothesis, rep-cap amplification was analyzed in the presence of an inhibitor of cellular polymerases. For this, HeRC32 cells were infected with wild type adenovirus for two hours. After this period, medium was changed and cells incubated with different concentrations of aphidicolin, a drug known to inhibit the activity of polymerases α, δ and ε. Two days later, DNA was analyzed by dot blot and hybridized either to a rep probe, to follow rep-cap amplification, or to a E2a probe, to follow the effect of the drug on adenovirus replication. As shown in FIG. 5B, the addition of aphidicolin strongly inhibited rep-cap amplification with a maximum effect reached at the concentration of 2.5 μg/ml. In contrast, aphidicolin did not inhibit adenovirus replication. Overall, these results indicated that cellular polymerase(s) were involved in the amplification process.

### Example 4

### Rep-Cap Amplification can be Induced in the Presence of DBP and Rep Proteins

Previous results indicated that the adenovirus E2b gene was not necessary for rep-cap amplification. To further investigate the role of adenovirus, the same analysis was perfomed using another adenoviral mutant harboring a thermosensitive mutation in the E2a gene encoding for the DBP (Ad.ts125).

As previously described, HeRC32 cells were infected with Ad.ts125 and maintained for 48 h at either 32° C. (permissive temperature) or 39° C. (non permissive temperature). Analysis of the rep-cap copy number by Southern blot indicated that amplification was severely reduced upon inactivation of the DBP (FIG. 5, lanes 4 and 5). This result suggested that this adenoviral factor might play a key role in the observed phenomenon. To confirm this hypothesis, a plasmid harboring the E2a gene under the control of the CMV promoter (CMVDBP) was transfected in HeRC32 cells six hours prior infection with Ad.ts125 at both the permissive and non-permissive temperatures. Analysis of rep-cap DNA 48 hours after infection revealed that rep-cap amplification could be restored to normal levels when cells were infected with Ad.ts125 at 39° C. and transfected with CMVDBP (FIG. 6, lanes 7 and 8).

To further validate the role of DBP in the amplification process, HeRC32 cells were transfected with the CMVDBP plasmid alone and analyzed for rep-cap copy number by Southern blot. A detectable level of amplification was seen under this condition (FIG. 6A, lane 10). The relatively low level of amplification seen upon transfection of CMVDBP was likely due to the inefficient transfection of this plasmid in HeRC32 compared to the efficiency of adenovirus infection.

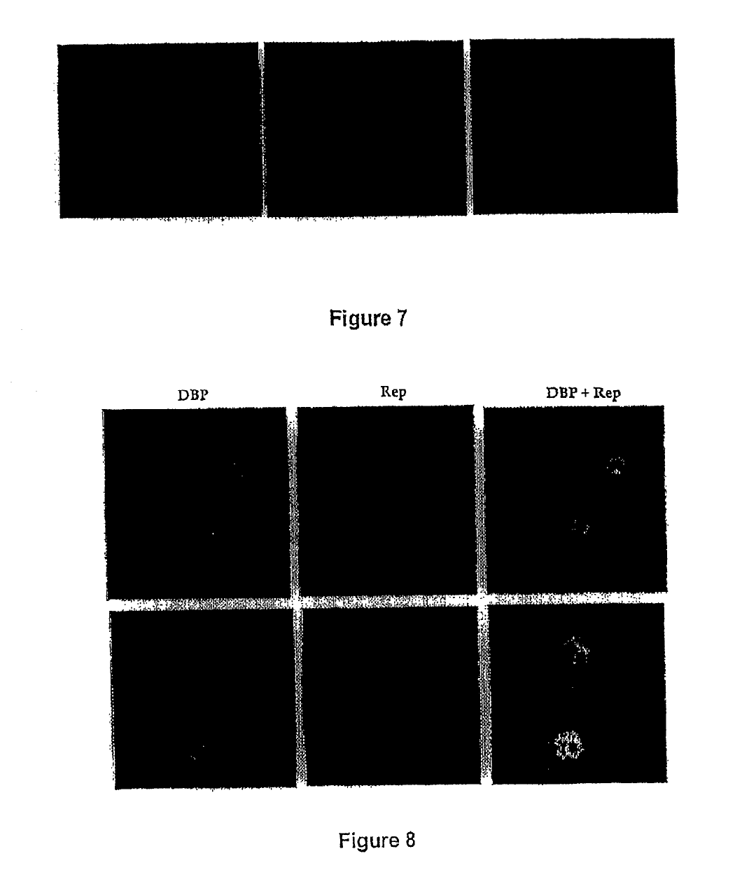

To verify this, HeRC32 cells transfected with the CMVDBP plasmid were analyzed by FISH to detect rep-cap amplification, As shown in FIG. 7 (panels A and B), an amplified rep-cap signal was detected in a small proportion of cells reflecting the overall transfection efficiency (approximately 5%). As previously observed in adenovirus-infected HeRC32 cells, it was not possible to visualize metaphases in cells displaying amplified rep-cap signal. No amplification was observed using a control plasmid (data not shown). These results indicated that among the adenovirus genes, the one encoding the DBP was sufficient to support rep-cap amplification.

### Example 5

### The ITR-Deleted Rep-Cap Genome is Packaged in AAV Capsids as Single-Stranded DNA

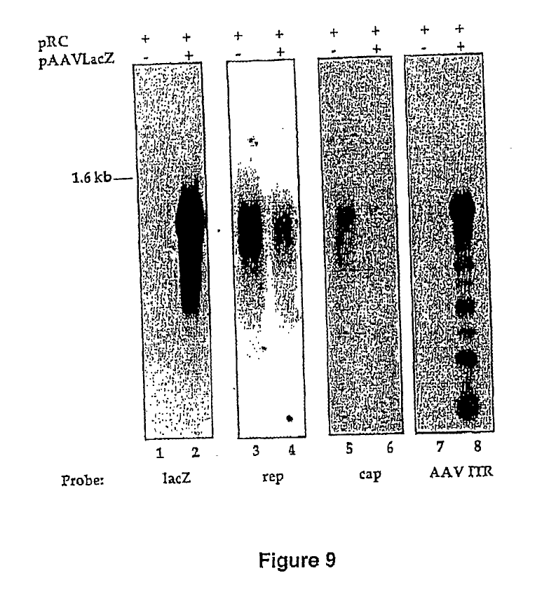

To determine if the rep-cap genome could be packaged in AAV particles in the absence of the ITRs, 293 cells were transfected with plasmid pRC, that contains the ITR-deleted rep-cap genome, either alone or in the presence of the pAAVLZ plasmid encoding the AAV vector, and then infected with adenovirus. At the time of the cytopathic effect, AAV particles were purified and viral DNA extracted. To prevent contamination with plasmid DNA, the particles were extensively treated with DNase I prior to proteinase K and phenol-chloroform DNA extraction. The DNA recovered under these conditions was analyzed on a Southern blot using a lacZ, rep, cap or AAV ITR probe (FIG. 9). In the presence of the pAAVLZ vector, a signal hybridizing to the rep and cap probes was detected as a smear (FIG. 9, lanes 4 and 6), indicating that, as reported in previous studies [10], rep-cap containing particles were generated. The key observation was that, in the absence of the pAAVLZ vector, rep and cap sequences were still detectable (FIG. 9, lanes 3 and 5). Identical results were obtained using a plasmid in which the ITR-deleted rep-cap genome was inserted in a different plasmid backbone (pSP72) (data not shown), thus excluding the influence of the latter. Two observations established the single-stranded nature of these rep-cap molecules. First, the hybridization signals were smears such as the one observed for AAVLZ DNA (FIG. 9, lane 2) and also migrated at a similar position in the gel. Second, and more importantly, the DNA analyzed in this experiment was transferred under neutral conditions and as such, the hybridization signal was restricted to single-stranded DNA. Indeed, the use of an adenovirus ITR probe confirmed that, under these conditions, double-stranded adenovirus DNA was not detected (data not shown). Importantly, wild type AAV contamination was also ruled out because: 1) the adenoviral stocks and the cells were routinely tested by PCR for the lack of contaminating wild type AAV (data not shown); 2) using a probe specific to the whole AAV ITR, no hybridization signal was observed in the case of particles produced using the pRC plasmid alone, whereas a signal was obtained with DNA extracted from rAAVLZ particles (FIG. 9, compare lanes 7 and 8, respectively); 3) rep-cap particles generated in the absence of rAAV vector were not competent for replication as indicated by Southern blot analysis using a rep probe after sequential amplification on adenovirus-infected 293 cells (data not shown). Altogether, these results indicated that an ITR-deleted rep-cap genome could be packaged into AAV capsids as single-stranded DNA.

### Example 6

### Evidence for In Vivo Replication of the pRC Plasmid in the Presence of Adenovirus

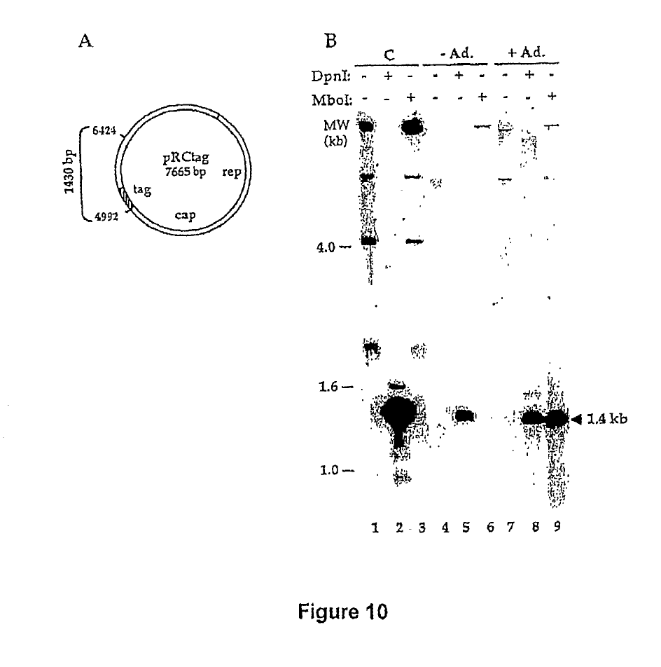

The result shown in example 5 implied that the pRC plasmid could replicate to generate single-stranded molecules. To verify this, 293 cells were first transfected with plasmid pRCtag harboring the rep-cap genome ligated to a tag sequence, and then mock or adenovirally-infected. After total DNA extraction, pRCtag plasmid replication was assessed by digestion with DpnI and MboI endonucleases, followed by Southern blot analysis using a tag probe. The activity of these enzymes depends upon the methylation pattern of the adenosines at their recognition sequence: cleavage by Dpn I indicates that both strands of the plasmid remains methylated in the absence of replication of the transfected DNA; cleavage by Mbo I occurs ouly if both strands are un-methylated, as a result of two rounds of replication. The results obtained (FIG. 10) indicated that, in the absence of adenovirus, the transfected pRCtag plasmid barely replicates in 293 cells (FIG. 10, lanes 5 and 6). In contrast, upon adenoviral infection, a fraction of the plasmid DNA template becomes susceptible to digestion with Mbo I (FIG. 10, lane 9). The same results were obtained using a rep probe (data not shown). When the same samples were analyzed after digestion with Dpn 1, resistant bands were detected as weak high molecular signals (FIG. 10, lane 8). Overall, these results indicated that both strands of the plasmid harboring the ITR-deleted AAV genome have replicated in adenovirus-infected 293 cells.

### Example 7

### A 350 bp Region Encompassing the p5 Promoter is Essential for the Replication of Plasmid pRC

To identify which element(s) in the pRCtag plasmid was responsible for the above observations, the 5′ portion of the rep gene was analyzed because it includes a RBS (nt 260-284 of wild type AAV) and a cryptic trs-like motif (nt 287 of wild type AAV). The presence of the same elements in the viral ITR is known to be essential for wild type AAV replication.

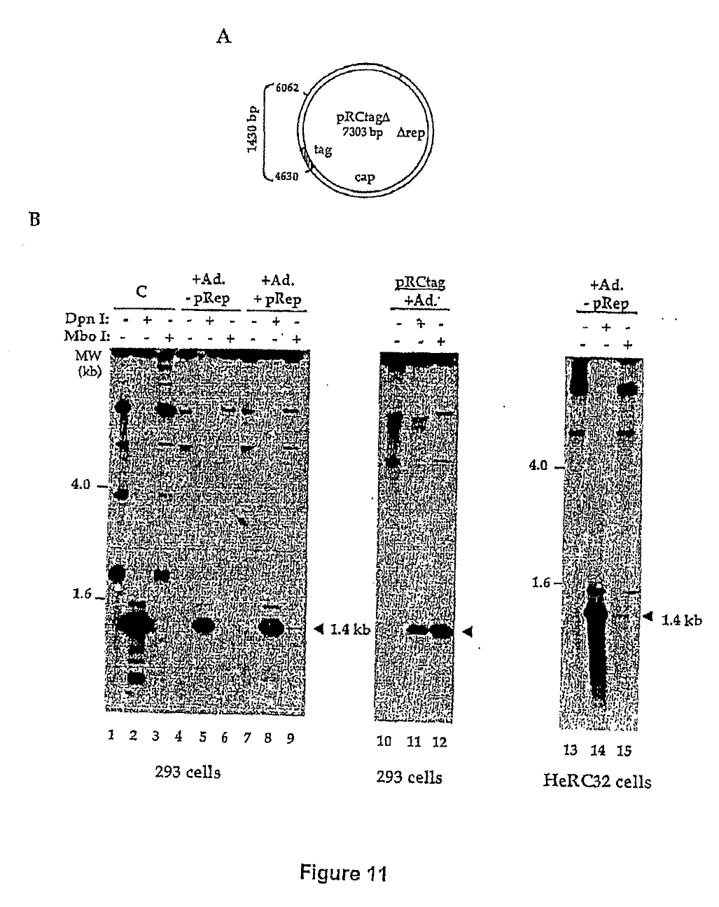

To evaluate the role of these nucleotide motifs on pRC replication, a rep-cap plasmid containing a 350 bp deletion in the 5′ portion of the rep gene (positions 191 to 540 of wild type AAV) was generated (pRCtag/Δ). This deletion, which removed both the RBS and the trs-like elements, extended from the p5 promoter into the 5′ coding sequence of the Rep78/68 ORFs. As a consequence, the pRCtag/Δ plasmid no longer produced Rep78 and Rep68. 293 cells were transfected with pRCtag/Δ either alone or with the pRep plasmid, to provide Rep in trans, and were subsequently infected with wild type adenovirus. Following total DNA extraction, replication of the input pRCtag/Δ plasmid was assessed by digestion with Dpn I and M.bo I endonucleases, as described above. The important result was that, even in the presence of both adenovirus and Rep proteins, the susceptibility of plasmid pRCtag/Δ to Mbo I digestion was severely reduced (FIG. 11, lane 9), compared to the level of replication of pRCtag under similar conditions (FIG. 11, lane 12). This result indicated that, upon deletion of the 350 nt region in the 5′ portion of the rep gene, replication of both strands of the ITR-deleted AAV genome was impaired. The same results were obtained in HeRC32 cells, which harbors one integrated copy of the intact ITR-deleted AAV-2 genome [7], suggesting that the cell background was not interfering (FIG. 11, lanes 13-15).

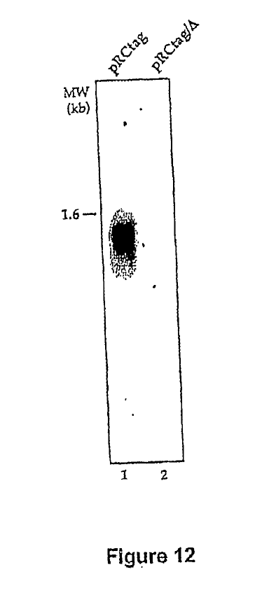

The consequence of this 350 bp deletion on the encapsidation of single-stranded rep-cap genomes into AAV particles was then evaluated. For this, HeRC32 cells were transfected with either pRCtag or pRCtag/Δ and then infected with wild type adenovirus. The HeRC32 cells were used to reduce the occurrence of recombination events between the pRCtag/Δ plasmid and the integrated wild type rep gene. Viral DNA was extracted from purified particles and analyzed by Southern blot using a tag probe. Results, presented FIG. 12, indicated that pRCtag generated single-stranded DNA which was encapsidated in AAV particles, thereby reproducing data described in FIG. 9 for plasmid pRC. However, upon transfection of the pRCtag/Δ plasmid, a hybridization signal was no longer detected (FIG. 12, lane 2). These results indicated that this 350 nt deletion in the rep gene which impaired replication also prevented encapsidation of single-stranded rep-cap sequences, despite functional Rep proteins provided in trans and adenovirus helper functions. Because of its effect on replication, this 350 nt region was further designated as a cis-acting Replication Element (CARE).

### Example 8

### CARE Behaves In Vivo and In Vitro as a Rep-dependent Origin of Replication

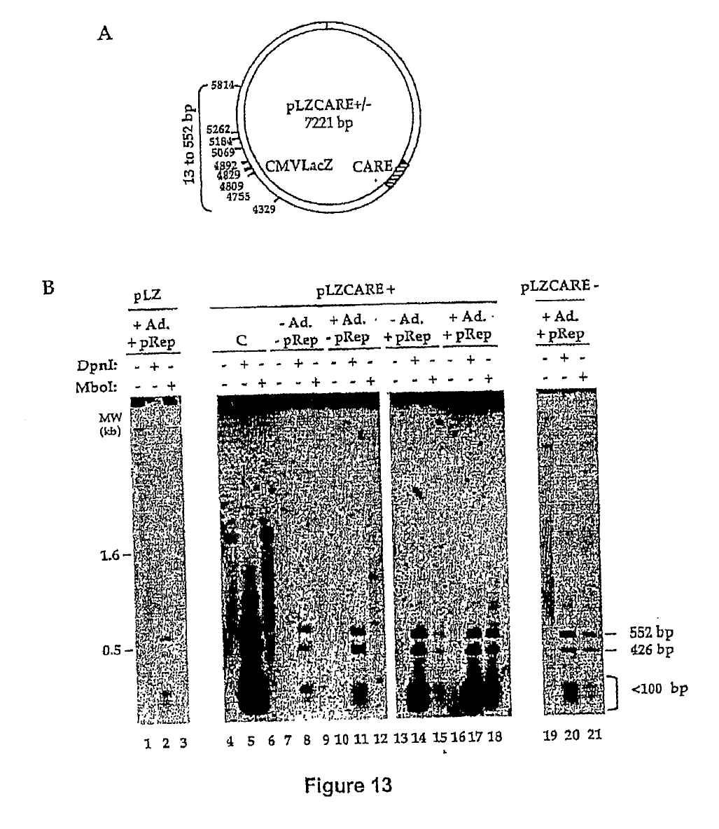

To further evaluate the ability of CARE to initiate DNA replication, this region was cloned in both orientations upstream of a heterologous sequence, the LacZ gene linked to the CMV immediate early promoter (FIG. 13A, plasmids pLZCARE+ (sense) and pLZCARE− (antisense)). These plasmids were transfected, alone or in combination with the pRep plasmid, into 293 cells that were subsequently infected with adenovirus or mock infected. Total DNA was digested with either DpnI or MboI and analyzed on a Southern blot using a LacZ probe (FIG. 13B). When DNA was extracted from cells transfected with a control plasmid (pLZ), no digestion products were detected, even in the presence of both adenovirus and Rep proteins (FIG. 13, lane 3). In contrast, both pLZCARE+ and pLZCARE− plasmids replicated, as shown by the detection of Mbo I digestion products in the presence of both Rep proteins and adenovirus (FIG. 13, lanes 18 and 21). In the absence of Rep proteins or adenovirus, no digestion products were detected (FIG. 13, lanes 9 and 12). The same result was reproduced with pLZCARE-plasmid (data not shown). As shown previously with the pRCtag plasmid, residual level of replication occurred upon co-transfection of the pRep plasmid alone (FIG. 13, lane 15). This most likely resulted from the synthesis of low levels of Rep proteins in 293 cells even in the absence of adenovirus infection. Overall, these results indicated that CARE could function as a Rep-dependent origin of replication in vivo.

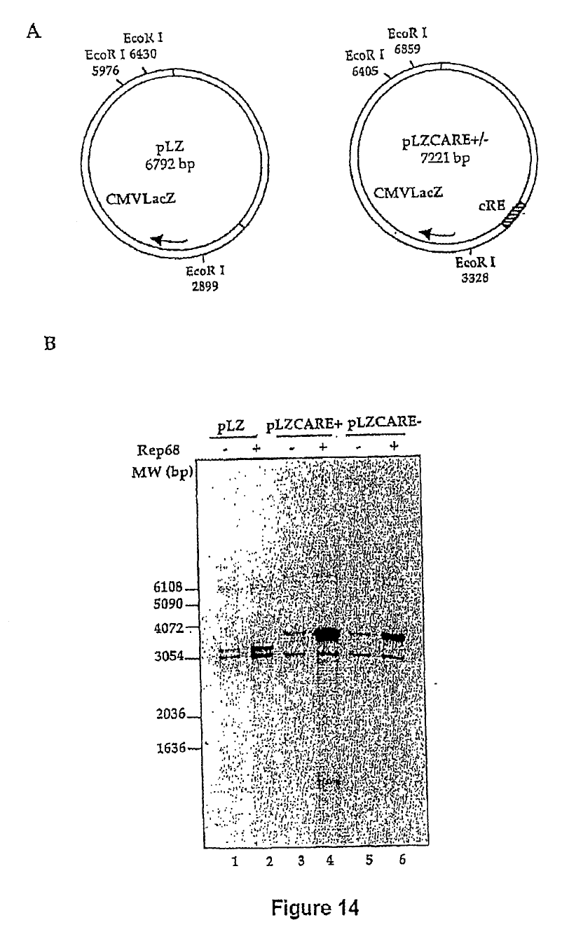

To validate this critical observation, in vitro replication experiments were performed. The substrate used in these reactions was the pLZ, pLZCARE+, or pLZCARE− plasmids previously digested with EcoR 1 (FIG. 14A). This enzyme generated two major linear DNA fragments, one containing the lacZ gene, corresponding to the lower band of FIG. 14B, and the upper band corresponding to the rest of the plasmid backbone associated, or not, with the CARE sequence. Reactions were performed using cell extracts from non-infected cells in the presence or absence of purified Rep68. The results indicated that a significant level of DNA repair occurred with every single plasmid, despite three hours of pre-incubation in the absence of labeled nucleotides. However, the important observation is that upon addition of purified Rep68, incorporation of labeled dCTP specifically increased in the upper band containing the CARE sequence for both plasmid pLZCARE+ and pLZCARE− (FIG. 14B, lanes 4 and 6). Quantification using a Phosphoimager indicated a 13 and 8.5-fold increase in incorporation for the CARE+ and the CARE− fragments, respectively. Altogether, these results demonstrated that CARE behaved in vivo and in vitro as a Rep-dependent origin of replication.

### Example 9

### The Herpesvirus Efficiently Induces Rep-Cap Replication in HeLa32 Cells

In order to compare the efficiency of wild-type HSV and various HSV mutants for their ability to induce rep-cap replication, HeRC32 cells were infected at different MOIs with:

wild-type HSV-1 (HSV-1-F WT) (MOIs of 0.5, 1, 5, 10 and 20 pfu/cell);

replication-defective mutants HSV-1 ΔICP4 (HSV-1-17 Cgal del IE3, [21]) (MOI of 1 pfu/cell); HSV-1 ΔICP27 (HSV-1-KOS 5dl1.2, [22]) (MOIs of 0.5, 1, 5, 10 and 20 pfu/cell);

an attenuated mutant HSV-1 ΔICP0 (HSV-1-17 dl1403, [23]) (MOIs of 0.5, 1 and 25 pfu/cell).

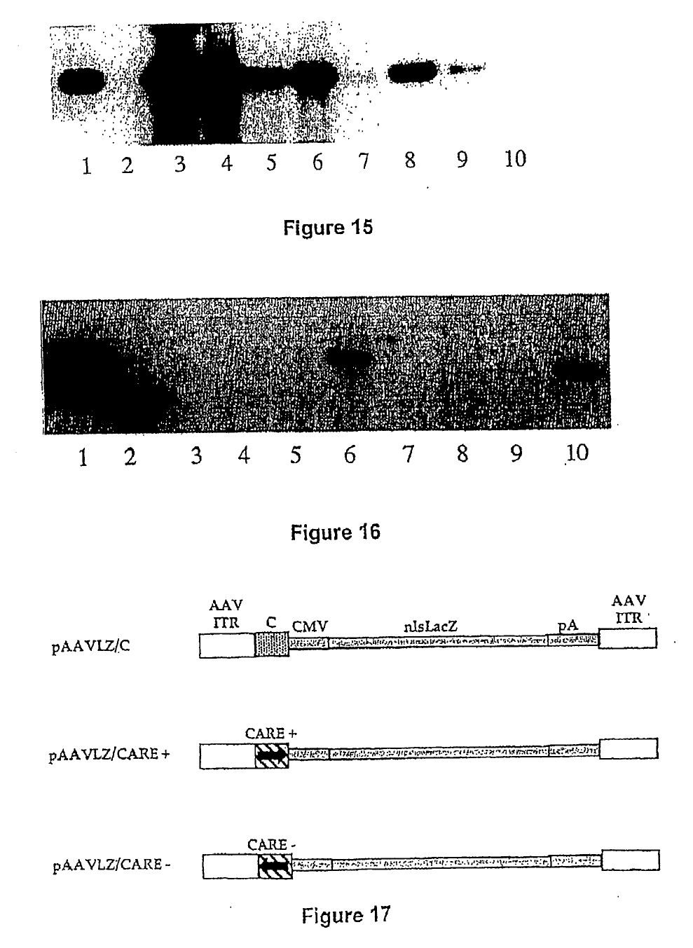

Infected cells were harvested 48 to 72 hours post infection and the amount of rep-cap copies estimated by Southern blot using a rep-cap probe. Cells infected with wild-type HSV-1 (FIG. 15), as well as with the mutants (results not shown), exhibited a strong amplification of the rep-cap signal, even superior in certain conditions to that observed in the presence of wild-type adenovirus. Indeed, herpesvirus infection at MOI=1 pfu/cell led to amplification yields comparable to those obtained when the cells are infected with adenovirus at MOI=50 pfu/cell (Compare lanes 1 and 6 of FIG. 15). Herpesvirus infection at higher MOIs (10 and 20 pfu/cell, for example) led to a stronger rep-cap amplification (at least 300-fold, as shown in FIG. 15, lanes 3 and 4). The fact that Herpesvirus infection could trigger rep-cap amplification more efficiently than wild-type adenovirus was completely unexpected.

These results demonstrate that HSV is able to amplify an integrated rep-cap genome like adenovirus. Moreover, these results show that ICP4, ICP27 and ICP0 proteins are not necessary for the amplification.

### Example 10

### rAAV Production Using Herpesvirus as Helper in HeRC32 Cells

The Herpesviruses referred to in Example 9 were then tested for rAAV production. HeLa32 cells were transfacted with pAAVCMVGFP, a plasmid harbouring a vector genome to be encapsidated. HSV infection was performed 6 hours later, rAAV production was estimated in the cell lysate by dot blot (titer in viral particles 1 ml) and by RCA (titer in infectious particles/ml).

rAAV production has been observed with wild type HSV (6×10¹⁰ particles/ml versus 1×10¹⁰ particles/ml with adenovirus), ΔICP27 and ΔICP0 mutants. However, ΔICP4 does not produce rAAV.

The use of the attenuated ΔICP0 mutant has never been described before. These data indicate that rAAV production could be performed in HeRC32 cells, using ΔICP27 or ΔICP0 in which a rAAV vector genome has been inserted by homologous recombination or direct cloning.

### Example 11

### CARE can Induce the Replication of a Heterologous Sequence

CARE-lacZ is a plasmid comprising a CARE sequence situated upstream from a CMV-nlsLacZ expression cassette. This plasmid has been integrated in the genome of HeLa cells, and stable cell clones have been obtained. In order to dertermine whether the presence of CARE could lead to the amplification of the LacZ gene, the cells were transfected with the plasmid Rep.pA, encoding rep proteins, and then infected with wild-type adenovirus. The cells were harvested 48 hours post infection, and total genomic DNA was extracted. The amount of LacZ copies was measured by Southern blot using a LacZ probe. The results (FIG. 16), show that the initial number of LacZ copies is very low, and that LacZ is significantly amplified in the presence of Rep and Adenovirus (compare lanes 3 and 7 versus 6 and 10, respectively). The same result was obtained using wild-type HSV (result not shown).

This result shows the implication of CARE in the amplification observed in examples 1 to 10 in the presence of Rep proteins and adenovirus or HSV. CARE could therefore be used for amplifying and hence over-expressing a gene X associated to CARE, in the presence of Rep proteins and a CARE-dependent replication inducer (CARE-DRI), selected for example from the group of Ad DBP, adenovirus and herpesvirus.

### Example 12

### CARE is Comprised in a 171 nt Sequence Corresponding to Nucleotides 190 to 361 of AAV-2

As shown in Example 7, CARE is comprised in a sequence derived from a fragment of the genome of wild-type AAV-2, including nucleotides 190 to 540 of AAV-2. A shorter sequence, corresponding to nucleotides 190 to 361 of AAV-2, and comprising the RBS and trs signals, was inserted upstream from the CMVLacZ expression cassette, generating the CARE.A.LacZ sequence. In vitro replication of this plasmid was evidenced in the presence of Rep68. To the contrary, the nucleotide sequence consisting of nucleotides 361 to 540 (referred to as CARE.B) could no longer enable Rep-dependent replication. Therefore, the CARE activity can be attributed to region A (nucleotides 190 to 360).

### Example 13

### The Presence of CARE in rAAV Vectors Increases rAAV Titers

The results shown in Examples 6, 7 8 and 11 demonstrate that CARE confers replication ability to heterologous plasmids. Finally, the effect of CARE was evaluated in the context of an AAV vector, where both ITRs are present.

For this, CARE was inserted into an AAVCMVLacZ vector, between the 5′ ITR and the CMV promoter (FIG. 17). Importantly, CARE was cloned in either the sense (pAAVLZ/CARE+) or the antisense orientation (pAAVLZ/CARE−). In pAAVLZ/CARE+, CARE Is located 3 nt closer to the 5′ ITR than the corresponding wild type AAV sequence. As a control, an unrelated sequence (C) of approximately the size of CARE was introduced in the same position in the AAVCMVLacZ vector (pAAVLZ/C). Consequently, the three rAAV vectors had similar sizes (4810 to 4860 bp ITR to ITR).

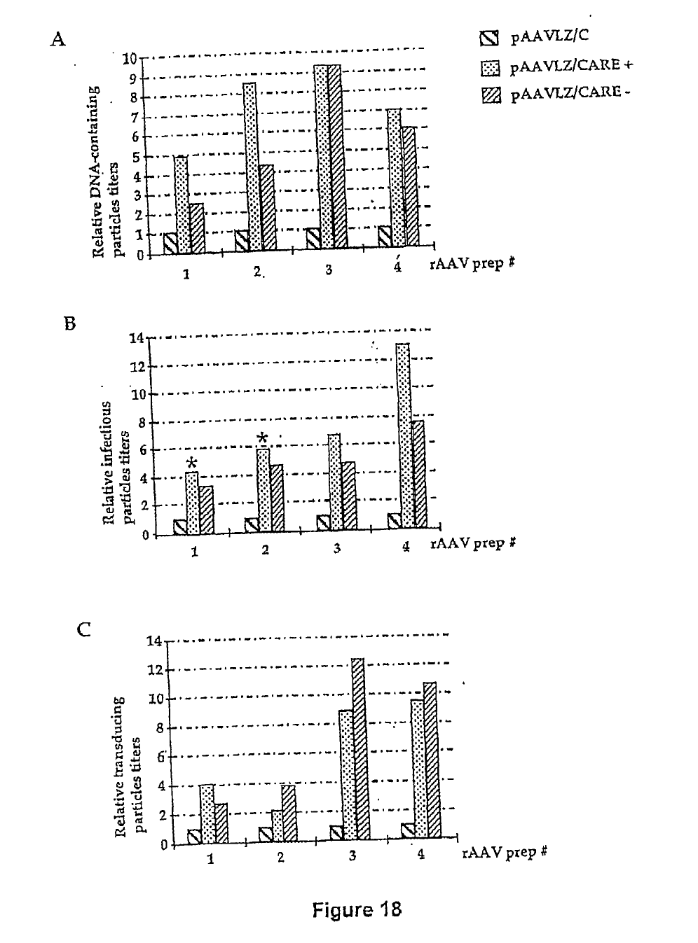

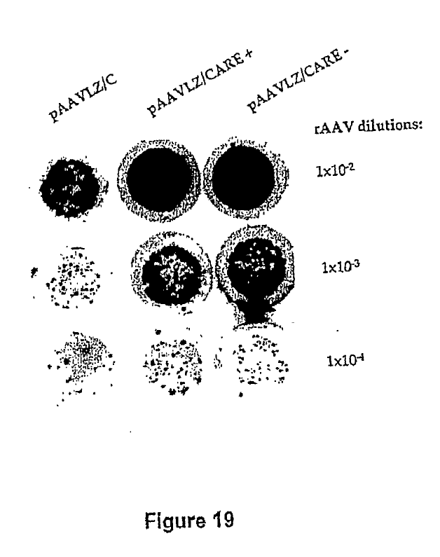

The three rAAV vectors (pAAVLZ/C, pAAVLZ/CARE+, and pAAVLZ/CARE−) were transfected into HeRC32 cells, which were then infected with wild type adenóvirus. Recombinant AAV particles were titrated either after purification on a CsCl gradient (stocks # 2, 3, and 4) or directly using the crude cell extract (stock # 1). FIG. 18 represents rAAV titers after arbitrarily setting to 1 the titers obtained from the control rAAVLZ/C stock. The data indicated that insertion of CARE in the rAAV vector resulted, on average, in a 6-fold increase in titers, irrespective of the orientation of CARE and of the titration method used. A typical example obtained by titrating rAAV infectious particles by mRCA [10] is presented in FIG. 19. Also, as expected, 0.01% rep-positive AAV particles were generated when CARE was present in the rAAV vector in the sense orientation, most likely through homologous recombination events between the vector containing CARE, and the endogenous rep-cap sequence present in the HeRC32 cells. Interestingly, these contaminating particles were no longer detected when CARE was inserted in the antisense orientation (pAAVLZ/CARE−). In conclusion, these data demonstrated that re-insertion of CARE into a rAAV vector in an appropriate orientation resulted in approximately a 6-fold increase in rAAV titers with no detectable rep-positive AAV contamination.

### Example 14

### rAAV Production Using a Clone Derived From HeRC32 Cells and Bearing an Integrated Recombinant AAV-CARE-eGFP Vector Genome

pAAVCARECMVeGFP is a plasmid comprising a CARE sequence, in antisense orientation, situated upstream from a CMV-eGFP expression cassette, the resulting CARECMVeGFP sequence being inserted between AAV-2 ITRs.

This plasmid comprises the following elements:

| | |
|---|---|
| ITR 5′ | 1→173 |
| CARE | 174→564 |
| prom. CMV | 565→990 |
| eGFP | 991→1758 |
| WPRE | 1759→2408 |
| BGH pA | 2409→2811 |
| ITR 3′ | 2812→2979, wherein |

prom. CMV designates the cytomegalovirus immediate early promoter, eGFP is the coding sequence for the green fluorescent protein, WPRE means “woodchuck post regulatory element”, and BGH pA corresponds to the polyadnenylation site of the bovine growth hormone gene.

A stable clone derived from HeRC32 cells and harboring an integrated copy of pAAVCARECMVeGFP was isolated (HeRC32/AAVCAREeGFP).

These cells were infected either by wild-type adenovirus or by wild-type HSV-1 or a mutant as described in Example 9. Forty-eight hours post infection, the cells were harvested and analyzed for

i) rep-cap amplification, by Southern blot;

ii) rAAVCAREeGFP production, by dot blot and RCA.

Rep-cap amplification was observed whatever the helper virus used. rAAV production was observed with both of the wild-type viruses and with ΔICP27 and ΔICP0 mutants, but not with the ΔICP4 mutant: However, since ΔICP27 is toxic for the cells, the rAAV production using this helper virus is not very efficient.

Importantly, this result shows that a rAAV vector comprising CARE and stably integrated in the cell genome does not interfere with the rep-cap amplification that takes place in the presence of a helper virus (adenovirus or herpesvirus). Moreover, this result demonstrates that when Rep and Cap proteins are present, as well as a helper virus, an AAV vector containing CARE can be efficiently excised and encapsidated.

The insertion of CARE in an AAV-derived vector, either in the form of a plasmid or a recombinant virus, seems also to facilitate the stable integration of these sequences.

### Example 15

### HeRC32/AAVCAREeGFP Producing Cells are Stable and Lead to High rAAV Titers Using Adenovirus or Herpesvirus as Helper Virus

HeRC32/AAVCAREeGFP cells have been cultured during more than one year and no rearrangement has been noticed. In particular, the integrated rAAV genome remained unchanged, as well as the rep-cap insert.

The producing efficiency of these cells was extensively studied, using either Adenovirus or Herpesvirus as CARE-DRI. The obtained rAAV preparations have been compared to preparations obtained using standard conditions, i.e., co-transfection of 293 cells by the plasmid pDG, which provides the rep and cap genes and the adenoviral necessary helper functions [24], and pAAVGFP, which carries the vector genome.

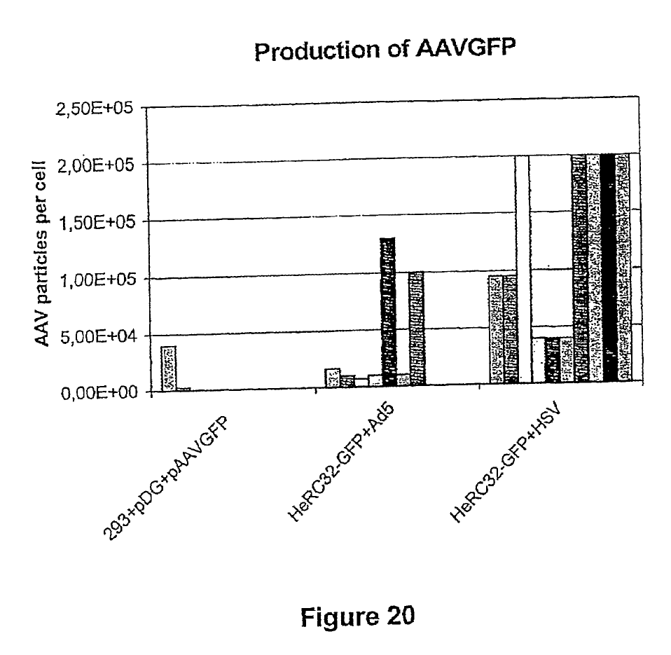

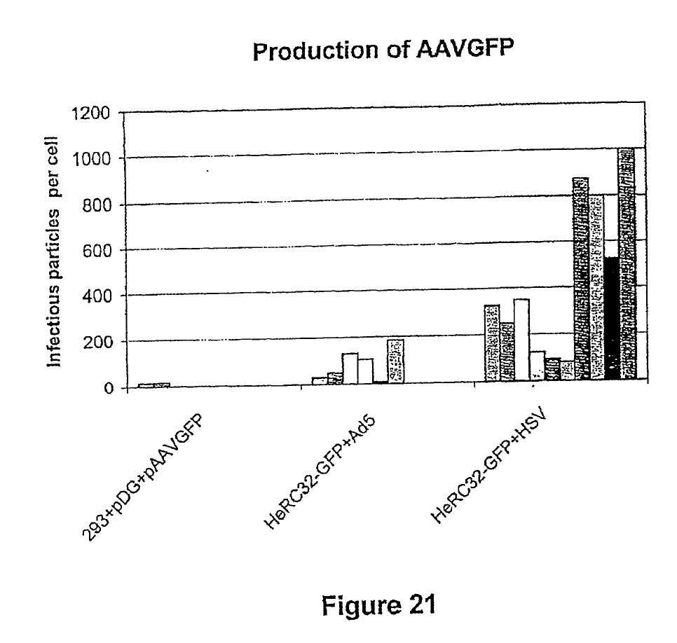

FIGS. 20 and 21 show the results (in viral particles/cell and in infectious particles/cell, respectively) of the following production experiments:

2 individual experiments in standard co-transfection conditions,

7 independent production experiments in HeRC32/AAVCAREeGFP cells using the Adenovirus as CARE-DRI. HeRC32/AAVCAREeGFP have been infected with wild-type Ad5 at a multiplicity of infection (MOI) of 50, and harvested forty-eight hours post infection; and

10 independent production experiments in HeRC32/AAVCAREeGFP cells using wild-type HSV-1 as CARE-DRI, at a MOI of 1. In these experiments, the cells have been collected 24 hrs post-infection.

The AAVGFP production rate (either in viral particles/cell or in infectious particles/cell) is 5 to 10-fold more efficient in HeRC32/AAVCAREeGFP cells infected with Adenovirus than in standard conditions, and even higher in HeRC32/AAVCAREeGFP cells infected with Herpesvirus. Interestingly, no Rep positive AAV particles were detected in the obtained preparations.

This example thus illustrates the great interest of stably integrating a rAAV genome comprising a CARE sequence into the genome of a producing cell, since the vector genome is here correctly mobilised and efficiently packaged.

It should be noted here that the skilled artisan can possibly further improve these results by performing an optimisation of one or several of the following parameters: confluence of the cells prior to infection by the CARE-DRI, MOI of infection, duration of infection prior to collecting the cells, means of recuperation of the rAAV particles, and the like.

### Example 16

### HeRC32/AAVCARE-LZ Producing Cells

HeRC32 cells were transfected with pAAVCARECMV-LZ, which is a plasmid analogous to pAAVCARECMVeGFP, except that:

the eGFP coding region is replaced by the LacZ coding region,

the CARE sequence used is shorter, and corresponds to region A (nucleotides 190 to 360) mentioned in Example 12, and

it does not contain the WPRE sequence.

A few clones derived from HeRC32 cells and harbouring an integrated copy of pAAVCARECMV-LZ were isolated and are examined for rAAV production.

### Example 17

### Comparison of Several CARE-DRIs for rAAV Production in HeRC32/AAVCAREeGFP Cells

As shown in Example 15, wild-type HSV-1 is a very efficient helper for rAAV production in cells harbouring a stable copy of a rAAV vector genome.

Different herpesvirus mutants have then been tested for their ability to induce CARE-dependent replication in order to produce rAAV from such producing cells. The use of defective mutants can be interesting for safety reasons. Six mutants were tested in HeRC32/AAVCAREeGFP cells, in the same conditions as mentioned in Example 15 for HSV-1, in three different experiments.

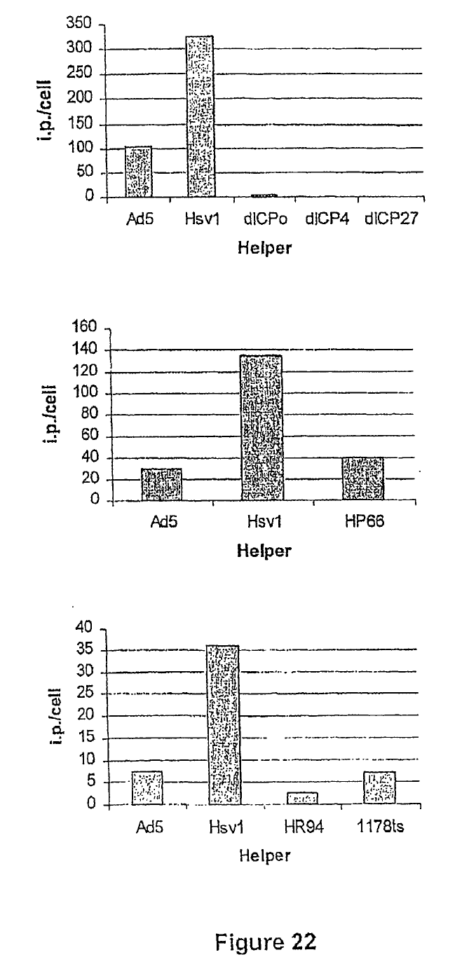

The result of these experiments is shown in FIG. 22.

The first experiment compares wild-type Ad5 and wild-type HSV-1 to replication-defective mutants HSV-1 ΔICP4 [21], and HSV-1 ΔICP27 [22], and to the attenuated mutant HSV-1 ΔICP0 [23]. rAAV production was observed with the ΔICP0 mutant, but not with the ΔICP4 mutant; the cytotoxicity of ΔICP27 led to cell death prior to efficient virus production.

The second set of experiments compares wild-type Ad5 and wild-type HSV-1 to the HSV mutant HP66 [25], which proves to be an interesting CARE-DRI, as least as efficient as wild-type Ad5.

The last experiments test the efficiency of two other HSV mutants, namely HR94 [26] and 1178ts [27].

### BIBLIOGRAPHY

(1) Dubielzig, R., et al., Adeno-associated virus type 2 protein interactions: formation of pre-encapsidation complexes. J Virol, 1999. 73(11): p.8989-98.

(2) Ward, P. and R. M. Linden, A role for single-stranded templates in cell-free adeno-associated virus DNA replication. J Virol, 2000. 74(2): p. 744-54.

(3) Clark, K. R., et al., Cell lines for the production of recombinant adeno-associated virus. Hum Gene Ther, 1995. 6(10): p. 132941.

(4) Inoue, N. and D. W. Russell, Packaging cells based on inducible gene amplification for the production of adeno-associated virus vectors. J Virol, 1998. 72(9): p. 7024-31.

(5) Gao, G. P., et al., High-titer adeno-associated viral vectors from a Rep/Cap cell line and hybrid shuttle virus. Hum Gene Ther, 1998. 9(16): p. 2353-62.

(6) Liu, X. L., K. R. Clark, and P. R. Johnson, Production of recombinant adeno-associated virus vectors using a packaging cell line and a hybrid recombinant adenovirus. Gene Ther, 1999. 6(2): p. 293-9.

(7) Chadeuf, G., et al., Efficient recombinant adeno-associated virus production by a stable rep-cap HeLa cell line correlates with adenovirus-induced amplification of the integrated rep-cap genome [in Process Citation]. J Gene Med, 2000. 2(4): p. 260-8.

(8) Liu, X., et al., Selective rep-Cap gene amplification as a mechanism for high-titer recombinant AAV production from stable cell lines [In Process Citation]. Mol Ther, 2000. 2(4): p. 394-403.

(9) Grimm, D., et al., Titration of AAV-2 particles via a novel capsid ELISA: packaging of genomes can limit production of recombinant AAV-2. Gene Ther, 1999. 6(7): p. 1322-30.

(10) Salvetti, A., et al., Factors influencing recombinant adeno-associated virus production. Hum Gene Ther, 1998. 9(5): p. 695-706.

(11) Meissner, J. D., Nucleotide sequences and further characterization of human papillomavirus DNA present in the CaSki, SiHa and HeLa cervical carcinoma cell lines. J Gen Virol, 1999. 80(Pt 7): p. 1725-33.

(12) Walz, C., et al., Interaction of human papillomavirus type 16 and adeno-associated virus type 2 co-infecting human cervical epithelium. J Gen Virol, 1997. 78(Pt 6): p. 1441-52.

(13) Samulski, R. J., L. S. Chang, and T. Shenk, Helper-free stocks of recombinant adeno-associated viruses: normal integration does not require viral gene expression. J Virol, 1989. 63(9): p. 3822-8.

(14) Klessig, D. F., D. E. Brough, and V. Cleghon, Introduction, stable integration, and controlled expression of a chimeric adenovirus gene whose product is toxic to the recipient human cell. Mol Cell Biol, 1984. 4(7): p. 1354-62.

(15) Ensinger, M. J. and H. S. Ginsberg, Selection and preliminary characterization of temperature-sensitive mutants of type 5 adenovirus. J Virol, 1972. 10(3): p. 328-39.

(16) Graham, F. L. and L. Prevec, Manipulation of adenovirus vectors. In E. J. Murray (ed.), Gene Transfer and Protocol. The Humana Press, Inc., Clifton, N.J., 1991: p. 109-128.

(17) Timbury, M. C., Temperature-sensitive mutants of herpes simplex virus type 2. J Gen Virol, 1971. 13(2): p. 373-6.

(18) Reich, N. C., et al., Monoclonal antibodies which recognize native and denatured forms of the adenovirus DNA-binding protein. Virology, 1983. 128(2): p. 480-4.

(19) Wistuba, A., et al., Intermediates of adeno-associated virus type 2 assembly: identification of soluble complexes containing Rep and Cap proteins. J Virol, 1995. 69(9): p. 5311-9.

(20) Ward, P., et al., Role of the adenovirus DNA-binding protein in in vitro adeno-associated virus DNA replication. J Virol, 1998. 72(1): p. 420-7.

(21) Johnson, P. A., et al., Isolation of a herpes simplex virus type I mutant deleted for the essential UL42 gene and characterization of its null phenotype. J Virol, 1991. 65(2): p. 700-10.

(22) McCarthy, A. M., L. McMahan, and P. A. Schaffer, Herpes simplex virus type 1 ICP27 deletion mutants exhibit altered patterns of transcription and are DNA deficient. J Virol, 1989. 63(1): p. 18-27.

(23) Stow, N. D. and E. C. Stow, Isolation and characterization of a herpes simplex virus type 1 mutant containing a deletion within the gene encoding the immediate early polypeptide Vmw110. J Gen Virol, 1986. 67(Pt 12): p. 2571-85.

(24) Grimm, D., et al., Novel tools for production and purification of recombinant adenoassociated virus vectors. Hum Gene Ther, 1998. 9(18): p. 2745-60.

(25) Marcy, A. I., D. R. Yager, and D. M. Coen, Isolation and characterization of herpes simplex virus mutants containing engineered mutations at the DNA polymerase locus. J Virol, 1990. 64(5): p. 2208-16.

(26) Malik, A. K., et al., Genetic analysis of the herpes simplex virus type 1 UL9 gene: isolation of a LacZ insertion mutant and expression in eukaryotic cells. Virology, 1992. 190(2): p. 702-15.

(27) Weller, S. K., et al., Genetic and phenotypic characterization of mutants in four essential genes that map to the left half of HSV-1 UL DNA. Virology, 1987.161(1): p. 198-210.

What is claimed is:

1. An isolated nucleic acid sequence comprising a first DNA sequence comprising a cis-acting replication element (CARE) from an Adeno-Associated Virus (AAV), and a second DNA sequence operably linked to said CARE, wherein amplification of said isolated nucleic acid sequence occurs when said isolated nucleic acid sequence is integrated in the genome of a cell and said cell is contacted with a CARE-dependent replication inducer (CARE-DRI).

2. The isolated nucleic acid sequence according to claim 1, wherein the nucleotide sequence of said CARE is the nucleotide sequence of SEQ ID N° 1 or a fragment thereof or a mutant of said fragment, provided said fragment or mutant still promotes the amplification of a DNA sequence integrated into the genome of a cell and operably linked to said CARE, following contacting said cell with a CARE-DRI.

3. The isolated nucleic acid according to claim 1 comprising a CARE and a polynucleotide sequence heterologous to AAV.

4. The nucleic acid of claim 3, further comprising a polylinker comprising several cloning sites.

5. The nucleic acid of claim 3, further comprising genetic elements from a virus.

6. The nucleic acid of claim 5, comprising retroviral Long Terminal Repeats (LTRs).

7. A method for the amplification of a DNA sequence in a cell, comprising the following steps:
(i) operably linking a DNA sequence to an isolated CARE;
(ii) introducing said sequence operably linked to the CARE into the cell genome; and
(iii) contacting said cell with a CARE-DRI.

8. The method of claim 7, wherein the cell is a cell-line harboring part of human papilloma virus selected from the group comprising HeLa, HeRC32, SIHA, CASKI cells and cells derived from HeLa, HeRC32, SIHA, and CASKI cells.

9. A method for the amplification of a DNA sequence operably linked to a CARE and integrated into the genome of a cell, comprising the step of contacting said cell with a CARE-DRI.

10. The method of claim 9, wherein the cell is a stable cell-line derived from human cells harboring part of human papilloma virus selected from the group comprising HeLa, HeRC32, SIHA and CASKI cells.

11. The method of any of claims 7 and 9, wherein the CARE-DRI is selected from the group comprising Adenoviruses, Herpesviruses, the adenoviral DNA-Binding Protein (Ad DBP), the gene of the Ad DBP, and any gene transfer vector expressing the Ad DBP.

12. A method for the amplification of a DNA sequence operably linked to a CARE and integrated into the genome of a cell, comprising the step of contacting said cell with a CARE-DRI wherein the DNA sequence to be amplified encodes the cap genes of an Adeno-Associated Virus.

13. The method according to claim 12, wherein the DNA sequence to be amplified further encodes the rep genes of an Adeno Associated Virus.

14. A highly producing rAAV packaging cell-line comprising
an integrated copy of the rep and cap genes, operably linked to a CARE; and
an integrated copy of an AAV-derived vector, comprising a DNA sequence of interest flanked by AAV Inverted Terminal Repeats (ITRs);
wherein replication of the integrated rep and cap genes is inducible by a CARE-DRI.

15. The packaging cell-line of claim 14, wherein the AAV-derived vector comprises a CARE sequence, in sense or antisense orientation.

16. The packaging cell-line of claim 15, wherein the CARE linked to the integrated rep and cap genes is in sense orientation, and the CARE comprised in the integrated rAAV vector is in antisense orientation.

17. The packaging cell-line of claim 14, further comprising a second integrated copy of the cap gene operably linked to a CARE sequence.

18. A highly producing rAAV packaging cell-line comprising
an integrated copy of the rep and cap genes, operably linked to a CARE sequence; and
a second integrated copy of the cap gene.

19. The packaging cell-line of claim 18, wherein the second integrated copy of the cap gene is operably linked to a CARE sequence.

20. The packaging cell-line of claim 14 or 18, which is derived from a human cell-line harbouring part of human papilloma virus such as HeLa, HeRC32, SIHA and CASKI cells.

21. A cell-line comprising an integrated CARE sequence operably linked to a DNA sequence heterologous to AAV and to the cells from which the cell-line is derived.

22. The cell-line of any of claims 14, 18 and 21, wherein one or several of the integrated elements is flanked by retroviral Long Terminal Repeats (LTRs).

23. A method of producing recombinant AAV preparations, comprising the step of contacting cells harboring rep and cap genes operably linked to a CARE sequence with a CARE-DRI.

24. The method of claim 23, wherein the cells are a cell-line according to claims 14 or 18.

25. A method of producing recombinant AAV preparation, comprising the steps of transfecting cell-line according to claim 18 with a plasmid harboring a rAAV genome comprising a CARE, and contacting said cell-line with a CARE-DRI.

26. The method of claim 23 or 25, wherein the CARE-DRI is selected from the group comprising Adenoviruses, Herpesviruses, the adenoviral DNA-Binding Protein (Ad DBP), the gene of the Ad DBP, and any gene transfer vector expressing the Ad DBP.

27. The method of claim 26, wherein said CARE-DRI is a herpesvirus.

28. The method of claim 27, wherein said CARE-DRI is a herpesvirus mutant from the group comprising ΔICP0, HP66, HR94, and 1178ts.

29. A kit for amplifying a DNA sequence in a cell, comprising a nucleic acid according to claim 1 and a CARE-DRI.

30. The kit of claim 29, wherein the CARE-DRI is selected from the group comprising Adenoviruses, Herpesviruses, the adenoviral DNA-Binding Protein (Ad DBP), the gene of the Ad DBP, and any gene transfer vector expressing the Ad DBP.

31. The kit of claim 29, further comprising a rep expression cassette.

32. The kit of claim 31, wherein said rep expression cassette is enclosed in a plasmid or in a vector selected from the group comprising Adenoviruses, Herpesviruses and Retroviruses.

33. The kit of claim 29, further comprising a purified Rep protein.


SEQUENCE LISTING (1 sequence)

SEQ ID NO: 1 - DNA, 24 bp, Artificial Sequence
  Description of Artificial Sequence Synthetic oligonucleotide
gatctctagtcagttaggcctccg